(19) United States
(12) Patent Application Publication
(10) Pub. No.: US 2025/0185909 A1
(43) Pub. Date: Jun. 12, 2025

(54) OPHTHALMIC INFORMATION PROCESSING APPARATUS, OPHTHALMIC APPARATUS, OPHTHALMIC INFORMATION PROCESSING METHOD, AND RECORDING MEDIUM

(71) Applicant: TOPCON CORPORATION, Tokyo (JP)

(72) Inventor: Riku KUJI, Tokyo (JP)

(73) Assignee: TOPCON CORPORATION, Tokyo (JP)

(21) Appl. No.: 18/969,293

(22) Filed: Dec. 5, 2024

(30) Foreign Application Priority Data
Dec. 8, 2023 (JP) 2023-207593

(51) Int. Cl. A61B 3/10; A61B 3/00

(52) U.S. Cl. CPC A61B 3/102; A61B 3/0025; A61B 3/0058

(57) ABSTRACT

An ophthalmic information processing apparatus includes an acquisition unit, a segmentation processor, and a display controller. The acquisition unit is configured to acquire an OCT image and one or more tomographic information images. The OCT image is a tomographic image of an eye to be examined. The one or more tomographic information images are generated using a method different from a method of generating the OCT image and represent tomographic information of the eye to be examined. The segmentation processor is configured to perform segmentation processing on the OCT image to identify a boundary of a layer region in a depth direction. The display controller is configured to display the OCT image, in which the boundary identified by the segmentation processor is distinguishably depicted, and the one or more tomographic information images on a display means.

### CROSS-REFERENCE TO RELATED APPLICATION

This application is based upon and claims the benefit of priority from Japanese Patent Application No. 2023-207593, filed Dec. 8, 2023; the entire contents of which are incorporated herein by reference.

### FIELD

The disclosure relates to an ophthalmic information processing apparatus, an ophthalmic apparatus, an ophthalmic information processing method, and a recording medium.

### BACKGROUND

Optical coherence tomography (OCT) apparatuses that are used to form images representing the surface morphology or the internal morphology of an object to be measured using light beam emitted from a laser light source or the like have been known. OCT performed in the OCT apparatuses is not invasive on the human body, and therefore is expected to be applied to the medical field or the biological field, in particular. For example, in the ophthalmic field, apparatuses for forming images of the fundus, the cornea, or the like have been in practical use. Such apparatuses using a method of OCT (OCT apparatuses) can be applied to observe tomographic structure of various sites of an eye to be examined. In addition, because of the ability to acquire high-definition images, the OCT apparatuses are applied to the diagnosis of various eye diseases.

In order to observe the tomographic structure of the eye to be examined, it is useful to perform segmentation (region division) processing on OCT images acquired using OCT to identify the layer regions that make up the tomographic structure. For example, the relationship between the depth in a depth direction of one or more specific layer regions and diseases is known, and the analysis of the thickness of the layer regions can be used as a biomarker. For example, by generating en-face images of desired one or more layer regions, the state of blood vessels or photoreceptor cells in the region can be observed in detail.

Various methods for segmentation have been proposed. Japanese Patent No. 7362403 discloses a method of suitably setting the region of interest using segmentation results for OCT images or OCT angiography (OCTA) images. Japanese Unexamined Patent Application Publication (Translation of PCT Application) No. 2023-524052 discloses a method of determining the segmentation quality of OCT structural data using OCTA flow data.

### SUMMARY

One aspect of embodiments is an ophthalmic information processing apparatus including: an acquisition unit configured to acquire an OCT image and one or more tomographic information images, the OCT image being a tomographic image of an eye to be examined, the tomographic information images being generated using a method different from a method of generating the OCT image and representing tomographic information of the eye to be examined; a segmentation processor configured to perform segmentation processing on the OCT image to identify a boundary of a layer region in a depth direction; and a display controller configured to display the OCT image, in which the boundary identified by the segmentation processor is distinguishably depicted, and the one or more tomographic information images on a display means.

Another aspect of the embodiments is an ophthalmic apparatus including: an optical system configured to perform OCT on an eye to be examined; an image forming unit configured to form an OCT image based on a detection result of interference light acquired by the optical system; and the ophthalmic information processing apparatus described above.

Still another aspect of the embodiments is an ophthalmic information processing method including: an acquisition step of acquiring an OCT image and one or more tomographic information images, the OCT image being a tomographic image of an eye to be examined, the tomographic information images being generated using a method different from a method of generating the OCT image and representing tomographic information of the eye to be examined; a segmentation processing step of performing segmentation processing on the OCT image to identify a boundary of a layer region in a depth direction; and a display control step of displaying the OCT image, in which the boundary identified by the segmentation processor is distinguishably depicted, and the one or more tomographic information images on a display means.

Still another aspect of the embodiments is a computer readable non-transitory recording medium in which a program for causing a computer to execute each step of the ophthalmic information processing method described above is recorded.

### BRIEF DESCRIPTION OF THE DRAWINGS

FIG. 1 is a schematic diagram illustrating an example of a configuration of an optical system of an ophthalmic apparatus according to embodiments.

FIG. 2 is a schematic diagram illustrating an example of the configuration of the optical system of the ophthalmic apparatus according to the embodiments.

FIG. 3 is a schematic diagram illustrating an example of a configuration of a processing system of the ophthalmic apparatus according to the embodiments.

FIG. 4 is a schematic diagram illustrating an example of the configuration of the processing system of the ophthalmic apparatus according to the embodiments.

FIG. 5 is a schematic diagram illustrating an example of the configuration of the processing system of the ophthalmic apparatus according to the embodiments.

FIG. 6 is an explanatory diagram of an operation of the ophthalmic apparatus according to the embodiments.

FIG. 7 is an explanatory diagram of the operation of the ophthalmic apparatus according to the embodiments.

FIG. 8 is an explanatory diagram of the operation of the ophthalmic apparatus according to the embodiments.

FIG. 9 is an explanatory diagram of an operation of the ophthalmic apparatus according to the embodiments.

FIG. 10 is an explanatory diagram of the operation of the ophthalmic apparatus according to the embodiments.

FIG. 11 is a flow chart of an example of an operation of the ophthalmic apparatus according to the embodiments.

FIG. 12 is a flow chart of an example of the operation of the ophthalmic apparatus according to the embodiments.

FIG. 13 is a flow chart of an example of the operation of the ophthalmic apparatus according to the embodiments.

FIG. 14 is a flow chart of an example of an operation of the ophthalmic apparatus according to the embodiments.

FIG. 15 is a flow chart of an example of the operation of the ophthalmic apparatus according to the embodiments.

### DETAILED DESCRIPTION

In segmentation processing for OCT images, it is often the case that the layer regions cannot be divided appropriately, depending on the image quality of the OCT image. In particular, in cases where the eye to be examined is a diseased eye, despite the need for more detailed observation, the fact is that it is often the case that the layer regions cannot be divided appropriately.

In this case, doctors, or the like will manually modify a boundary of a layer region identified by the segmentation processing. For example, when a plurality of slices of OCT imaging are performed with a raster scan, the doctors, or the like must manually modify the boundary of the layer region for each slice, so it takes a great deal of effort. In case that the image quality of the OCT image is low, it becomes even more difficult for the doctors, or the like to modify the boundary of the layer region with high accuracy.

As described above, under the current circumstances, it is sometimes difficult to identify the layer region in the tomographic structure of the eye to be examined with high accuracy.

According to some embodiments of the present invention, a new technique for identifying the layer region in the tomographic structure of the eye to be examined with high accuracy can be provided, while reducing a burden on a user.

Referring now to the drawings, exemplary embodiments of an ophthalmic information processing apparatus, an ophthalmic apparatus, an ophthalmic information processing method, and a program according to the present invention are described below. Any of the contents of the documents cited in the present specification and arbitrary known techniques may be applied to the embodiments below.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

An ophthalmic information processing apparatus according to embodiments includes an acquisition unit, a segmentation processor, and a display controller. The acquisition unit is configured to acquire an OCT image and one or more tomographic information images. The OCT image is a tomographic image of an eye to be examined. The one or more tomographic information images are generated using a method different from a method of generating the OCT image. Each of the tomographic information images represents tomographic information of the eye to be examined. The segmentation processor is configured to perform segmentation processing on the OCT image to identify a boundary of a layer region in a depth direction. The display controller is configured to display the OCT image, in which the boundary identified by the segmentation processor is distinguishably depicted, and the one or more tomographic information images described above on a display means.

In some embodiments, the acquisition unit is configured to acquire the OCT image and the one or more tomographic information images from outside the ophthalmic information processing apparatus via a network. That is, the ophthalmic information processing apparatus according to the embodiments may be configured to acquire the OCT image and the one or more tomographic information images from outside the ophthalmic information processing apparatus.

In some embodiments, the acquisition unit is configured to acquire the OCT image by performing OCT scan (OCT imaging, OCT measurement) on the eye to be examined using an optical system. In this case, an ophthalmic apparatus provided with the optical system realizes the function(s) of the ophthalmic information processing apparatus according to the embodiments.

In some embodiments, the acquisition unit is configured to acquire the one or more tomographic information images acquired by measuring (imaging, photographing) using OCT scan to the eye to be examined or a method different from the OCT scan, using an optical system. In some embodiments, the ophthalmic information processing apparatus is configured to acquire at least one of the OCT image or the one or more tomographic information images using the optical system, and to acquire remaining image(s) from an external source.

The tomographic information image is a tomographic image generated using a method different from a method of generating the OCT image and representing tomographic information of the eye to be examined. Examples of the tomographic information image include an OCTA image, an attenuation coefficient image, a polarization information image, a birefringence image, or a superimposed image of the above images. When any one of the OCTA image, the attenuation coefficient image, the polarization information image, and the birefringence image has been selected as a reference image, the superimposed image is an image in which one or more of the OCTA image, the attenuation coefficient image, the polarization information image, and the birefringence image, excluding the above reference image, are superimposed on the reference image. The tomographic information image is generated using a method different from a method of generating the OCT image. Thereby, the tomographic information image is tomographic information in which a distribution of the characteristics of physical quantities different from the reflection intensity at each position of the tomographic structure based on the backscattered light of the measurement light of OCT is imaged. Therefore, the tomographic information image may clearly depict the boundary of the layer region that is not distinctly depicted in the OCT image.

In particular, in case that the OCT image and the tomographic information image are generated by OCT scan or the tomographic information image is generated based on the OCT image, registration (position matching) between the OCT image and the tomographic information image becomes unnecessary. Thereby, the position in one of the OCT image and the tomographic information images can be easily identified from the position in the other image(s).

The boundary of the layer region according to the embodiments may be a linear (straight or curved) boundary demarcating two layer regions adjacent to each other in the depth direction, or a region, which has a width in the depth direction, demarcating two layer regions adjacent to each other in the depth direction.

Examples of depicting distinguishable boundary include depicting the boundary in a different color or brightness from other boundaries, depicting the boundary using a straight or curved line that is thicker (or thinner) than other boundaries, depicting the boundary that blinks in a manner different from other boundaries, and adding information (letters, arrows, etc.) indicating such boundary.

According to the embodiments, the boundary identified using the segmentation processing in the OCT image can be observed in detail while referring to one or more tomographic information images. For example, the position of the boundary identified by the segmentation processing can be observed or the above boundary can be modified, while referring to one or more tomographic information images. As a result, the layer region in the tomographic structure of the eye to be examined can be identified with high accuracy while reducing labor.

In some embodiments, the display controller is configured to display the OCT image, in which the boundary is depicted using the segmentation processing, and one of the one or more tomographic information images in a superimposed state on the display means. In some embodiments, the one or more tomographic information images are a plurality of tomographic information images. The display controller is configured to display the plurality of tomographic information images in a parallel or a superimposed state on the display means. In some embodiments, the boundary of the layer region in the depth direction is distinguishably depicted in the one or more tomographic information images. This makes it possible to easily identify the boundary of the layer region to be modified in the OCT image from the boundary of the layer region in one or more tomographic information images.

In some embodiments, the ophthalmic information processing apparatus includes an operation unit and a modification processor. The modification processor is configured to perform modification processing for modifying the boundary of the layer region in the OCT image, based on operation information of a user to the operation unit. The modification processing changes the positions of one or more pixels that make up the boundary of the layer region in the OCT image before modification based on operation information, and sets a boundary defined by one or more pixels whose positions have been changed as a new modified boundary of the layer region in the OCT image.

An ophthalmic information processing method is a method for controlling the ophthalmic information processing apparatus according to the embodiments. A program according to the embodiments causes a computer (processor) to execute each step of the ophthalmic information processing method according to the embodiments. In other words, the program according to the embodiments is a computer program comprising instructions which, when the program is executed by a computer, cause the computer to carry out the ophthalmic information processing method according to the embodiments. A recording medium (storage medium) according to the embodiments is any computer readable non-transitory recording medium (storage medium) on which the program according to the embodiments is recorded. The recording medium may be an electronic medium using magnetism, light, magneto-optical, semiconductor, or the like. Typically, the recording medium is a magnetic tape, a magnetic disk, an optical disk, a magneto-optical disk, a flash memory, a solid state drive, or the like. The computer program may be transmitted and received through a network such as the Internet, LAN, etc.

In this specification, the processor includes, for example, a circuit(s) such as, for example, a CPU (central processing unit), a GPU (graphics processing unit), an ASIC (application specific integrated circuit), and a PLD (programmable logic device). Examples of PLD include a simple programmable logic device (SPLD), a complex programmable logic device (CPLD), and a field programmable gate array (FPGA). The processor realizes, for example, the function according to the embodiments by reading out a computer program stored in a storage circuit or a storage device and executing the computer program. At least a part of the storage circuit or the storage apparatus may be included in the processor. Further, at least a part of the storage circuit or the storage apparatus may be provided outside of the processor.

Hereinafter, a case where an ophthalmic apparatus capable of acquiring an OCT image, which is a tomographic image of the eye to be examined, and a tomographic information image realizes the function(s) of the ophthalmic information processing apparatus according to the embodiments will be described. However, the ophthalmic information processing apparatus according to the embodiments may be provided outside the ophthalmic apparatus, and the ophthalmic information processing apparatus may be configured to acquire at least one of the OCT image (the tomographic image) or the tomographic information image from the ophthalmic apparatus.

Further, hereinafter, a case where the ophthalmic apparatus according to the embodiments is configured to acquire the OCT image and the one or more tomographic information images of the eye to be examined (subject's eye) by performing OCT on the eye to be examined will be described. This allows to observe the morphology of the tomographic structure of a desired site in the OCT image in detail, using any of the one or more tomographic information images, without performing registration processing on the OCT image and the one or more tomographic information images. It should be noted that the ophthalmic apparatus according to the embodiments may be configured to acquire at least one of the one or more tomographic information images from outside the ophthalmic apparatus. In this case, by performing registration processing on the OCT image and the tomographic information image(s) that is/are acquired from outside, the morphology of the tomographic structure of the desired site in the OCT image can be observed in detail using any of the one or more tomographic information images.

The ophthalmic apparatus according to the embodiments can perform OCT on an arbitrary site of the eye to be examined, such as the fundus, or the anterior segment, for example. In this specification, an image acquired using OCT may be collectively referred to as an “OCT image”. In this case, unless otherwise indicated, the OCT image will be explained as being the tomographic image. Also, the measurement operation for forming OCT image may be referred to as OCT measurement.

Hereinafter, in the embodiments, the case of using the swept source type OCT method in the measurement and the imaging (photographing) using OCT will be described. However, the configuration according to the embodiments can also be applied to an ophthalmic apparatus using other type of OCT (for example, spectral domain type OCT or time domain OCT).

### [Configuration]

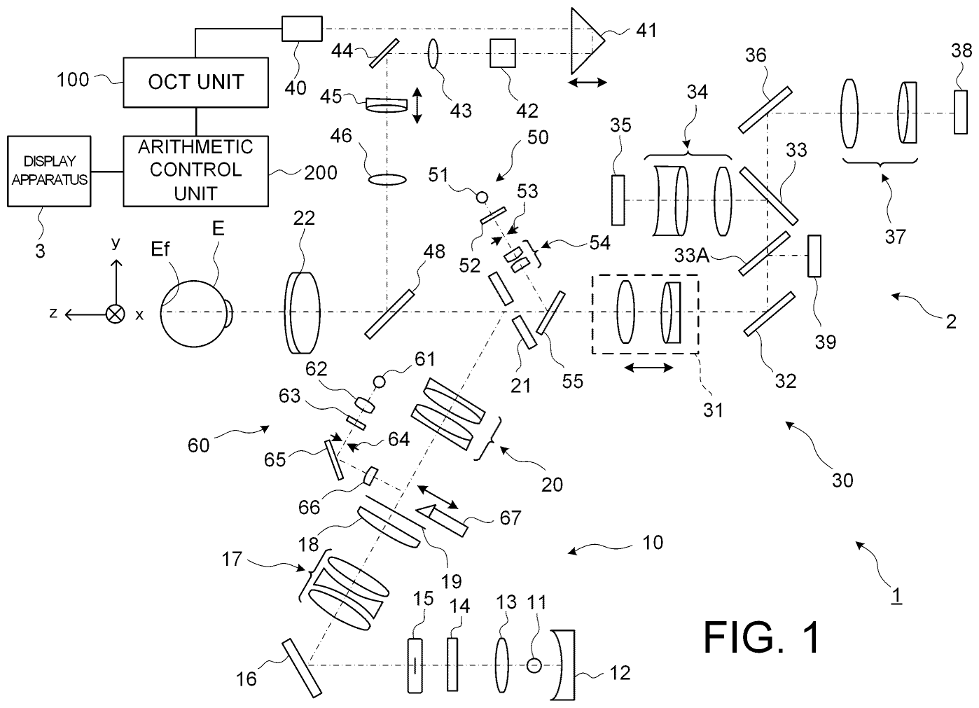

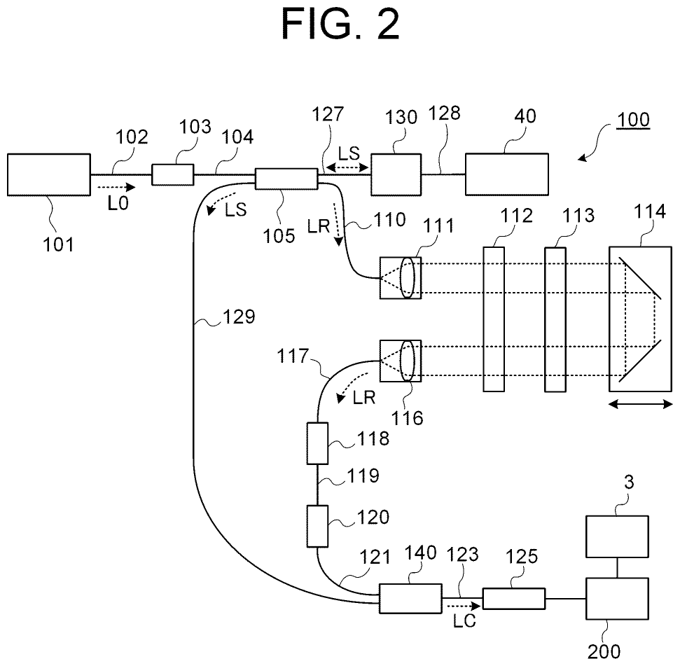

As shown in FIG. 1 and FIG. 2, an ophthalmic apparatus 1 according to the embodiments includes a fundus camera unit 2, an OCT unit 100, and an arithmetic control unit 200. The fundus camera unit 2 has substantially the same optical system as the conventional fundus camera. The OCT unit 100 is provided with an optical system for obtaining OCT images (for example, tomographic images) of the fundus (or the anterior segment). The arithmetic control unit 200 is provided a computer(s) that executes various kinds of arithmetic processing, control processing, and the like.

### [Fundus Camera Unit 2]

The fundus camera unit 2 illustrated in FIG. 1 is provided with an optical system for acquiring two-dimensional images (fundus images) representing the surface morphology of a fundus Ef of an eye E to be examined (subject's eye E). Examples of the fundus images include observation images and photographic images. The observation image is, for example, a monochrome moving image formed at a predetermined frame rate using near-infrared light. The photographic image may be, for example, a color image captured by flashing visible light, or a monochrome still image using near-infrared light or visible light as illumination light. The fundus camera unit 2 may be configured to be capable of acquiring other types of images such as fluorescein angiograms, indocyanine green angiograms, and autofluorescent angiograms.

The fundus camera unit 2 is provided with a jaw holder and a forehead rest for supporting the face of a subject (examinee). Further, the fundus camera unit 2 is provided with an illumination optical system 10 and an imaging optical system 30. The illumination optical system 10 irradiates illumination light onto the fundus Ef. The imaging optical system 30 guides the illumination light reflected from the fundus Ef to an imaging device (i.e., the CCD image sensor 35 or 38). Each of the CCD image sensors 35 and 38 is sometimes simply referred to as a “CCD”. Further, the imaging optical system 30 guides measurement light coming from the OCT unit 100 to the fundus Ef, and guides the measurement light via the fundus Ef to the OCT unit 100.

An observation light source 11 in the illumination optical system 10 includes, for example, a halogen lamp. Light (observation illumination light) emitted from the observation light source 11 is reflected by a reflective mirror 12 having a curved reflective surface, travels through a condenser lens 13, and becomes near-infrared light after passing through a visible cut filter 14. Further, the observation illumination light is once converged near an imaging light source 15, is reflected by a mirror 16, and passes through relay lenses 17 and 18, a diaphragm 19, and a relay lens 20. Then, the observation illumination light is reflected on the peripheral part (the surrounding area of the hole part) of the perforated mirror 21, is transmitted through a dichroic mirror 48, and refracted by the objective lens 22, thereby illuminating the fundus Ef. It should be noted that an LED (light emitting diode) may be used as the observation light source.

Fundus reflected light of the observation illumination light is refracted by the objective lens 22, is transmitted through the dichroic mirror 48, passes through the hole part formed in the center area of the perforated mirror 21, is transmitted through a dichroic mirror 55, travels through a focusing lens 31, and is reflected by a mirror 32. Further, this fundus reflected light is transmitted through a half mirror 33A, is reflected by a dichroic mirror 33, and forms an image on the light receiving surface of the CCD image sensor 35 by a condenser lens 34. The CCD image sensor 35 detects the fundus reflected light at a predetermined frame rate, for example. An image (observation image) based on the fundus reflected light detected by the CCD image sensor 35 is displayed on a display apparatus 3. It should be noted that when the imaging optical system 30 is focused on the anterior segment, an observation image of the anterior segment of the eye E to be examined is displayed.

The imaging light source 15 includes, for example, a xenon lamp. Light (imaging illumination light) emitted from the imaging light source 15 is irradiated onto the fundus Ef through the same route as that of the observation illumination light. The fundus reflected light of the imaging illumination light is guided to the dichroic mirror 33 via the same route as that of the observation illumination light, is transmitted through the dichroic mirror 33, is reflected by a mirror 36, and forms an image on the light receiving surface of the CCD image sensor 38 by a condenser lens 37. The display apparatus 3 displays an image (photographic image) obtained based on the fundus reflected light detected by the CCD image sensor 38. It should be noted that the display apparatus 3 for displaying the observation image and the display apparatus 3 for displaying the photographic image may be the same or different. Besides, when similar imaging is performed by illuminating the eye E to be examined with infrared light, an infrared photographic image is displayed. It is also possible to use an LED as the imaging light source.

A liquid crystal display (LCD) 39 displays a fixation target and a visual target used for visual acuity measurement. The fixation target is a visual target for fixating the eye E to be examined, and is used when performing fundus imaging (photography) and OCT measurement.

Part of light emitted from the LCD 39 is reflected by the half mirror 33A, is reflected by the mirror 32, travels through the focusing lens 31 and the dichroic mirror 55, and passes through the hole part of the perforated mirror 21. The light having passed through the hole part of the perforated mirror 21 is transmitted through the dichroic mirror 48, and is refracted by the objective lens 22, thereby being projected onto the fundus Ef.

By changing the display position of the fixation target on the screen of the LCD 39, the fixation position of the eye E to be examined can be changed. Examples of the fixation position of the eye E to be examined include a position for acquiring an image centered at a macular region of the fundus Ef, a position for acquiring an image centered at an optic disc, and a position for acquiring an image centered at the fundus center between the macular region and the optic disc. Further, the display position of the fixation target may be changed to any desired position.

In addition, as with a conventional fundus camera, the fundus camera unit 2 is provided with an alignment optical system 50 and a focus optical system 60. The alignment optical system 50 generates an indicator (an alignment indicator) for the position matching (alignment) of the optical system with respect to the eye E to be examined. The focus optical system 60 generates a target (split target) for adjusting the focus with respect to the eye E to be examined.

The light output from an LED 51 of the alignment optical system 50 (i.e., alignment light) travels through the diaphragms 52 and 53 and the relay lens 54, is reflected by the dichroic mirror 55, and passes through the hole part of the perforated mirror 21. The light having passed through the hole part of the perforated mirror 21 is transmitted through the dichroic mirror 48, and is projected onto the cornea of the eye E to be examined by the objective lens 22.

Cornea reflected light of the alignment light travels through the objective lens 22, the dichroic mirror 48 and the hole part described above. Part of the cornea reflected light is transmitted through the dichroic mirror 55, and passes through the imaging focusing lens 31, is reflected by the mirror 32, and is transmitted through the half mirror 33A. The cornea reflected light transmitted through the half mirror 33A is reflected by the dichroic mirror 33, and forms an image on the light receiving surface of the CCD image sensor 35 by the condenser lens 34. A light receiving image (an alignment indicator) captured by the CCD image sensor 35 is displayed on the display apparatus 3 together with the observation image. A user performs an alignment in the same manner as performed on a conventional fundus camera. Instead, alignment may be performed in such a way that the arithmetic control unit 200 analyzes the position of the alignment indicator and moves the optical system (automatic alignment).

To perform focus adjustment, a reflective surface of a reflection rod 67 is arranged in a slanted position on an optical path of the illumination optical system 10. The light output from a LED 61 in the focus optical system 60 (i.e., focus light) passes through a relay lens 62, is split into two light beams by a split indicator plate 63, passes through a two-hole diaphragm 64, and is reflected by a mirror 65. The focus light reflected by the mirror 65 is once converged on the reflective surface of the reflection rod 67 by the condenser lens 66, and is reflected by the reflective surface. Further, the focus light travels through the relay lens 20, is reflected by the perforated mirror 21, is transmitted through the dichroic mirror 48, and is refracted by the objective lens 22, thereby being projected onto the fundus Ef.

Fundus reflected light of the focus light passes through the same route as the cornea reflected light of the alignment light and is detected by the CCD image sensor 35. The display apparatus 3 displays the light receiving image (split indicator) captured by the CCD image sensor 35 together with the observation image. As in the conventional case, the arithmetic control unit 200 analyzes the position of the split indicator, and moves the focusing lens 31 and the focus optical system 60 for focusing (automatic focusing). Alternatively, the user may perform focusing manually while visually checking the split indicators.

The dichroic mirror 48 branches the optical path for OCT measurement from the optical path for fundus imaging (photography). The dichroic mirror 48 reflects light of wavelengths used for OCT measurement, and transmits light for fundus imaging. The optical path for OCT measurement is provided with, in order from the OCT unit 100 side, a collimator lens unit 40, an optical path length (OPL) changing unit 41, an optical scanner 42, a collimate lens 43, a mirror 44, an OCT focusing lens 45, and a field lens 46.

The optical path length changing unit 41 is configured to be capable of moving in a direction indicated by the arrow in FIG. 1, thereby changing the optical path length for OCT measurement. The change in the optical path length is used for the correction of the optical path length according to the axial length of the eye E to be examined, and/or for the adjustment of the interference state, or the like. The optical path length changing unit 41 includes, for example, a corner cube and a mechanism for moving the corner cube.

The optical scanner 42 is disposed at a position conjugate optically to a pupil of the eye E to be examined (pupil conjugate position) or near the position. The optical scanner 42 changes the traveling direction of light (measurement light) traveling along the optical path for OCT measurement. The optical scanner 42 can deflect the measurement light in a one-dimensionally or two-dimensional manner, under the control from the arithmetic control unit 200 described below.

The optical scanner 42 includes a first galvano mirror, a second galvano mirror, and a mechanism for driving them independently, for example. The first galvano mirror deflects measurement light LS so as to scan the imaging site (fundus Ef or the anterior segment) in a horizontal direction (x direction) orthogonal to an optical axis of the interference optical system. The second galvano mirror deflects the measurement light LS deflected by the first galvano mirror so as to scan the imaging site in the vertical direction (Y direction) orthogonal to the optical axis of the interference optical system. Thereby, the imaging site can be scanned with the measurement light LS in any direction on the x-y plane.

For example, by controlling an orientation of the first galvano mirror and an orientation of the second galvano mirror included in the optical scanner 42 at the same time, the irradiated position of the measurement light can be moved along an arbitrary trajectory on the x-y plane. This allows to scan the imaging site according to a desired scan pattern.

The OCT focusing lens 45 is movable along the optical path of the measurement light LS (the optical axis of the interference optical system). The OCT focusing lens 45 moves along the optical path of the measurement light LS, under the control from the arithmetic control unit 200 described below.

In some embodiments, a liquid crystal lens or an Alvarez lens is provided instead of the OCT focusing lens 45. The liquid crystal lens or the Alvarez lens, as well as the OCT focusing lens 45, is controlled by the arithmetic control unit 200.

### [October Unit 100]

The configuration of the OCT unit 100 will be described with reference to FIG. 2. The OCT unit 100 is provided with an optical system for performing OCT on the fundus Ef. In the present embodiment, this optical system can be used to acquire at least one of OCT images, OCTA images, attenuation coefficient images, DOPU (Degree Of Polarization Uniformity) images as polarization information images, or (and) birefringence images, as tomographic information images. That is, the optical system includes an interference optical system configured to split light from a wavelength scanning type (wavelength sweeping type) light source into measurement light and reference light, to make the measurement light returned from the fundus Ef and the reference light having passed through a reference optical path interfere with each other to generate interference light, and to detect the interference light. The detection result (detection signal) of the interference light obtained by the interference optical system is a signal indicating the spectra of the interference light and is sent to the arithmetic control unit 200.

Like the general swept source type OCT apparatus, a light source unit 101 includes a wavelength scanning type (wavelength sweeping type) light source capable of scanning (sweeping) the wavelengths of emitted light. The light source unit 101 temporally changes the output wavelengths within the near-infrared wavelength bands that cannot be visually recognized with human eyes.

The light L0 emitted from the light source unit 101 is guided to a polarization controller 103 through an optical fiber 102, and a polarization state of the light L0 is adjusted. The polarization controller 103, for example, applies external stress to the looped optical fiber 102 to thereby adjust the polarization state of the light L0 guided through the optical fiber 102.

The light L0 whose polarization state has been adjusted by the polarization controller 103 is guided to a fiber coupler 105 through an optical fiber 104, and is split into the measurement light LS and the reference light LR.

On the other hand, the measurement light LS generated by the fiber coupler 105 is guided to an incident polarization control unit 130 through an optical fiber 127. The incident polarization control unit 130 generates the measurement light LS with two polarization states whose polarization directions are orthogonal to each other or the measurement light LS with the two generated polarization states superimposed, from the incident measurement light LS. The measurement light LS with the two polarization states is the x-polarized (first polarization state) measurement light and the y-polarized (second polarization state) measurement light. The measurement light LS emitted from the incident polarization control unit 130 is guided to the collimator lens unit 40 through an optical fiber 128. The collimator lens unit 40 converts the incident measurement light LS into a parallel light beam. The measurement light LS made into the parallel light beam reaches the dichroic mirror 48 via the optical path length changing unit 41, the optical scanner 42, the collimate lens 43, the mirror 44, the OCT focusing lens 45, and the field lens 46. Subsequently, the measurement light LS is reflected by the dichroic mirror 48, is refracted by the objective lens 22, and is projected onto the fundus Ef. The measurement light LS is scattered and reflected (including reflection) at various depth positions of the fundus Ef. Back-scattered light of the measurement light LS from the fundus Ef reversely advances along the same path as the outward path, and is guided to the fiber coupler 105. Then, the back-scattered light passes through an optical fiber 129, and arrives at a polarization separation unit 140.

On the other hand, the reference light LR generated by the fiber coupler 105 is guided to the collimator 111 through an optical fiber 110 and becomes a parallel light beam. The reference light LR, which has become the parallel light beam, is guided to a corner cube 114 via an optical path length correction member 112 and a dispersion compensation member 113. The optical path length correction member 112 acts as a delay means for matching the optical path length (i.e., the optical distance) of the reference light LR and that of the measurement light LS. The dispersion compensation member 113 acts as a dispersion compensation means for matching the dispersion characteristic of the reference light LR and that of the measurement light LS.

The corner cube 114 reverses the traveling direction of the reference light LR that has become the parallel light beam by the collimator 111. The optical path of the reference light LR incident on the corner cube 114 and the optical path of the reference light LR emitted from the corner cube 114 are parallel to each other. Further, the corner cube 114 is movable in a direction along the incident light path and the emitting light path of the reference light LR. Through such movement, the optical path length of the reference light LR (i.e., the reference optical path) is varied.

The reference light LR that has traveled through the corner cube 114 passes through the dispersion compensation member 113 and the optical path length correction member 112, is converted from the parallel light beam to a convergent light beam by a collimator 116, and enters an optical fiber 117. The reference light LR that has entered the optical fiber 117 is guided to a polarization controller 118. With the polarization controller 118, the polarization state of the reference light LR is adjusted.

The polarization controller 118 has the same configuration as, for example, the polarization controller 103. The reference light LR whose polarization state has been adjusted by the polarization controller 118 is guided to an attenuator 120 through an optical fiber 119, and the light amount of the reference light LR is adjusted under the control of the arithmetic control unit 200. The reference light LR whose light amount has been adjusted by the attenuator 120 is guided to the polarization separation unit 140 through an optical fiber 121.

The polarization separation unit 140 separates the measurement light LS (returning light) incident through the optical fiber 129 into the measurement light LS (returning light) with two polarization states whose polarization directions are orthogonal to each other. The measurement light LS (returning light) with the two polarization states is the x-polarized measurement light (returning light) and the y-polarized measurement light (returning light). Subsequently, the polarization separation unit 140 combines (interferes) the measurement light LS and the reference light LR that has passed through the optical fiber 121 for each polarization state to generate interference light with two polarization states, or generates interference light in which the two generated polarization states are superimposed. In some embodiments, the polarization separation unit 140 is configured to separate the reference light LR into the reference light LR with two polarization states whose polarization directions are orthogonal each other, and then to generate the interference light between the returning light of the x-polarized measurement light LS and the x-polarized reference light LR and the interference between the returning light of the y-polarized measurement light LS and the y-polarized reference light LR.

The polarization separation unit 140 splits the interference light at a predetermined splitting ratio (e.g., 50:50) to generate a pair of interference light LC for each polarization state or a pair of interference light LC with two polarization states superimposed. The pair of interference light LC output from the polarization separation unit 140 is guided to the detector 125 through a light guiding member 123.

The detector 125 is, for example, a balanced photodiode that includes a pair of photodetectors for respectively detecting the pair of interference light LC and outputs the difference between the pair of detection results obtained by the pair of photodetectors. The detector 125 sends the detection result (i.e., detection signal) to the arithmetic control unit 200. For example, the arithmetic control unit 200 performs the Fourier transform etc. on the spectral distribution based on the detection result obtained by the detector 125 for each series of wavelength scanning (i.e., for each A-line) to form the tomographic image as the OCT image. The arithmetic control unit 200 displays the formed image on the display apparatus 3.

By emitting the pair of interference light LC with two polarization states superimposed from the polarization separation unit 140, the OCT image can be generated, for example, based on the detection result of the pair of interference light LC, which is obtained by the detector 125, with two polarization states superimposed. Alternatively, by emitting the pair of interference light LC for each of the two polarization states synthesized for each polarization state from the polarization separation unit 140, the OCT image can be generated, for example, based on the synthesis result obtained by further synthesizing the detection results of the interference light LC, which are obtained by the detector 125, with two polarization states. In this case, by controlling the incident polarization control unit 130, it is possible to configure so that the measurement light LS with two polarization states superimposed is generated.

The OCTA image can be generated, for example, using a plurality of OCT images acquired in the same manner as described above by repeatedly performing OCT scan on the same site. Alternatively, the OCTA image can be generated, for example, using the detection result of a plurality of chronological the pair of interference light LC with two polarization states superimposed acquired in the same way as described above by repeatedly performing OCT scan on the same site. The position of the site depicted in the OCTA image like this is determined with reference to one of the OCT images used for generating the OCTA image. Therefore, the registration processing between the OCTA image and the OCT images is unnecessary.

The attenuation coefficient image can be generated, for example, using the OCT image, as described below. Therefore, the registration processing between the attenuation coefficient image and the OCT image is unnecessary.

By emitting the interference light with two polarization states synthesized for each polarization state from the polarization separation unit 140, the DOPU image can be generated, for example, based on the detection result of the interference light with two polarization states obtained by the detector 125. In this case, by controlling the incident polarization control unit 130, it is possible to configure so that the measurement light LS with two polarization states superimposed is generated.

By emitting the measurement light LS with two polarization states from the incident polarization control unit 130 and emitting the interference light with two polarization states synthesized for each polarization state from the polarization separation unit 140, the birefringence image can be generated, for example, based on the detection result of the pair of interference light LC for each of the two polarization states obtained by the detector 125.

As described above, by emitting the measurement light LS with two polarization states from the incident polarization control unit 130, emitting the interference light with two polarization states synthesized for each polarization state from the polarization separation unit 140, and detecting the interference light with two polarization states obtained by the detector 125, the OCT image (tomographic image), the DOPU image, and the birefringence image can be acquired using a single OCT scan. Therefore, the registration processing among the OCT image, the DOPU image and the birefringence image can be made unnecessary. In addition, as described above, since the registration processing between the OCTA image and the OCT image can be made unnecessary, the registration processing among the OCT image, the OCTA image, the DOPU image, and the birefringence image can be made unnecessary.

Although a Michelson interferometer is employed in the present embodiment, it is possible to employ any type of interferometer such as Mach-Zehnder-type as appropriate. In the present embodiment, in addition to the configuration shown in FIG. 2, the interference optical system may further include the collimator lens unit 40, the optical path length changing unit 41, the optical scanner 42, the collimate lens 43, the mirror 44, the OCT focusing lens 45, and the field lens 46, which are shown in FIG. 1. This interference optical system is an example of the “interference optical system” according to the embodiments.

### [Arithmetic Control Unit 200]

The configuration of the arithmetic control unit 200 will be described.

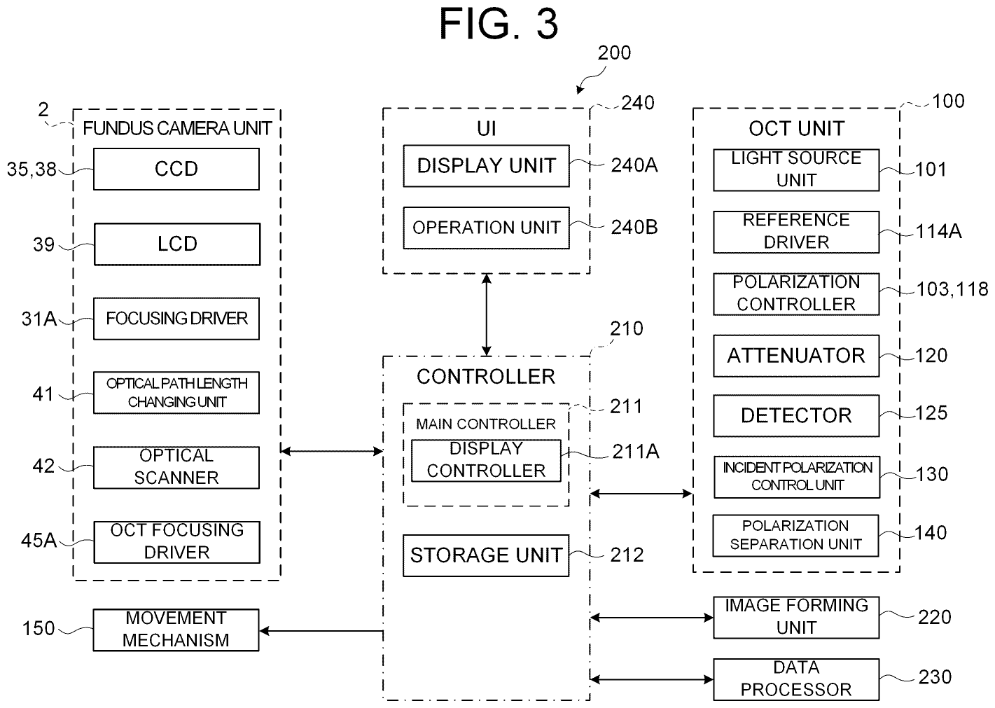

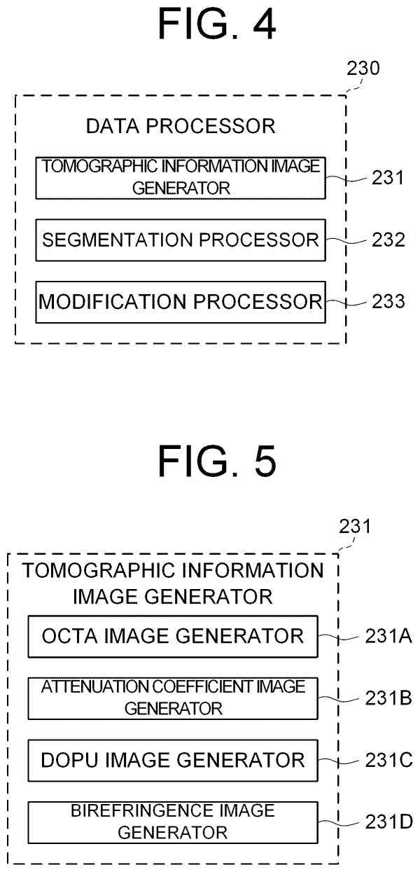

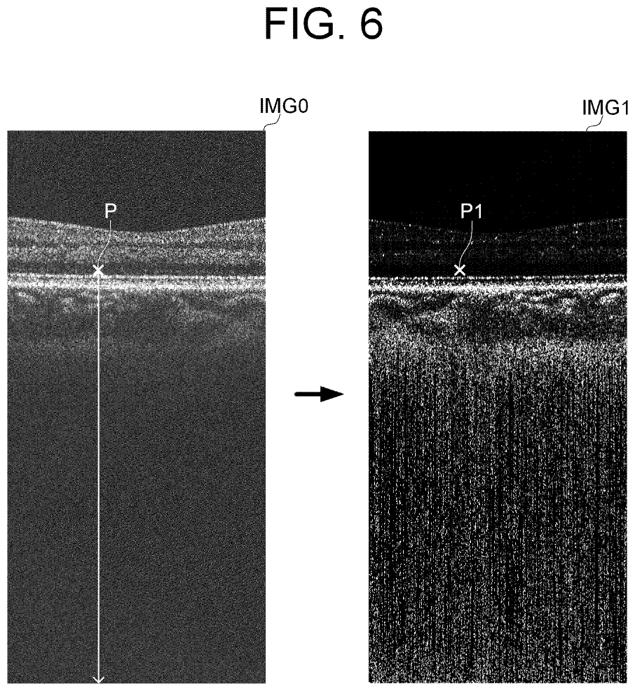

FIG. 3, FIG. 4, and FIG. 5 show block diagrams of examples of a configuration of a processing system (control system) of the ophthalmic apparatus 1 according to the embodiments. FIG. 4 shows a functional block diagram representing an example of a configuration of a data processor 230 in FIG. 3. FIG. 5 shows a functional block diagram representing an example of a configuration of a tomographic information image generator 231 in FIG. 3. In FIG. 3, FIG. 4, and FIG. 5, like reference numerals designate like parts as in FIG. 1 or FIG. 2. The same description may not be repeated.

The arithmetic control unit 200 analyzes the detection signals fed from the detector 125 to form at least one of the OCT image of the fundus Ef (or anterior segment), the OCTA image of the fundus Ef (or anterior segment), the attenuation coefficient image of the fundus Ef (or anterior segment), the DOPU image of the fundus Ef (or anterior segment), or the birefringence image of the fundus Ef (or anterior segment). The arithmetic processing for the OCT image formation is performed in the same manner as in the conventional swept source type ophthalmic apparatus.

As shown in FIG. 3, the arithmetic control unit 200 includes a controller 210, and controls each part of the fundus camera unit 2, the display apparatus 3, and the OCT unit 100. For example, the arithmetic control unit 200 forms at least one of the OCT image of the fundus Ef, the OCTA image of the fundus Ef, the attenuation coefficient image of the fundus Ef, the DOPU image, or the birefringence image of the fundus Ef, and displays the formed image(s) on the display apparatus 3 (display unit 240A described below).

Examples of the control for the fundus camera unit 2 include the operation control for the observation light source 11, the imaging light source 15, the LEDs 51 and 61, the operation control for the CCD image sensors 35 and 38, the operation control for the LCD 39, the movement control for the focusing lens 31, the movement control for the OCT focusing lens 45, the movement control for the reflection rod 67, the operation control for the alignment optical system 50, the movement control for the focus optical system 60, the movement control for the optical path length changing unit 41, and the operation control for the optical scanner 42.

Examples of the control for the OCT unit 100 include the operation control for the light source unit 101, the movement control for the corner cube 114, the operation control for the detector 125, the operation control for the attenuator 120, the operation control for the polarization controllers 103 and 118, the operation control for the incident polarization control unit 130, and the operation control for the polarization separation unit 140.

Like conventional computers, the arithmetic control unit 200 includes a microprocessor, a RAM (random access memory), a ROM (read only memory), a hard disk drive, a communication interface, and the like. A storage device such as the hard disk drive stores a computer program for controlling the ophthalmic apparatus 1. The arithmetic control unit 200 may include various kinds of circuitry such as a circuit board for forming OCT images. In addition, the arithmetic control unit 200 may include an operation device (or an input device) such as a keyboard and a mouse, and a display device such as an LCD. In some embodiments, the functions of the arithmetic control unit 200 are realized by one or more processors.

The fundus camera unit 2, the display apparatus 3, the OCT unit 100, and the arithmetic control unit 200 may be integrally provided (i.e., in a single housing), or they may be separately provided in two or more housings.

The controller 210 includes a main controller 211 and a storage unit 212.

### (Main Controller 211)

The main controller 211 performs various controls by outputting control signals to each part of the ophthalmic apparatus 1 described above. In particular, the main controller 211 controls components of the fundus camera unit 2 such as the CCD image sensors 35 and 38, the LCD 39, the focusing driver 31A, the optical path length changing unit 41, the optical scanner 42, and the OCT focusing driver 45A. Further, the main controller 211 controls components of the OCT unit 100 such as the light source unit 101, the reference driver 114A, the polarization controllers 103 and 118, the attenuator 120, the detector 125, the incident polarization control unit 130, and the polarization separation unit 140.

The main controller 211 controls an exposure time (charge accumulation time), a sensitivity, a frame rate, or the like of the CCD image sensor 35 or 38. In some embodiments, the main controller 211 controls the CCD image sensor 35 or 38 so as to acquire images having the desired image quality.

The main controller 211 performs display control of fixation targets or visual targets for the visual acuity measurement, for the LCD 39. Thereby, the visual target presented to the eye E to be examined can be switched, or type of the visual targets can be changed. Further, the presentation position of the visual target to the eye E to be examined can be changed by changing the display position of the visual target on the screen of the LCD 39.

The focusing driver 31A moves the focusing lens 31 in the optical axis direction. The main controller 211 controls the focusing driver 31A so that the focusing lens 31 is positioned at a desired focusing position. As a result, the focusing position of the imaging optical system 30 (returning light from the imaging site) is changed.

For example, the main controller 211 analyzes the position of the split indicator in the light receiving image obtained by the CCD image sensor 35, and controls the focusing driver 31A and the focus optical system 60. Alternatively, for example, the main controller 211 controls the focusing driver 31A and the focus optical system 60. according to the operations performed by the user to the operation unit 240B described below, while displaying a live image of the eye E to be examined on the display unit 240A described below.

The main controller 211 controls the optical path length changing unit 41 to change the optical path length of the measurement light LS. Thereby, the difference between the optical path length of the measurement light LS and the optical path length of the reference light LR is changed.

For example, the main controller 211 analyzes the detection result of the interference light LC obtained by OCT measurement (or the OCT image formed based on the detection result), and controls the optical path length changing unit 41 so that the measurement site is positioned at a desired depth position.

The main controller 211 is configured to control the optical scanner 42. The main controller 211 controls the optical scanner 42 so as to deflect the measurement light LS according to the deflection pattern corresponding to the scan mode set in advance.

Examples of scan mode like this include a line scan, a cross scan, a circle scan, a radial scan, a concentric scan, a multiline cross scan, a helical scan (spiral scan), a Lissajous scan, a three-dimensional scan, and an ammonite scan. The ammonite scan is a scan mode in which a scan reference position (scan center position) of the circle scan as a high-speed scan is moved along the scan pattern of the spiral scan as a low-speed scan. In other words, the circle scan is performed sequentially around each scan center position while moving the scan center position along the spiral scan pattern.

By scanning the imaging site with the measurement light LS according to the deflection pattern corresponding to the scan mode as described above, the tomographic image as the OCT image in the plane stretched by the direction along the scan line (scan trajectory) and the fundus depth direction (z direction) can be acquired.

The OCT focusing driver 45A moves the OCT focusing lens 45 along the optical axis of the measurement light LS. The main controller 211 controls the OCT focusing driver 45A so that the OCT focusing lens 45 is positioned at a desired focusing position. As a result, the focusing position of the measurement light LS is changed. The focusing position of the measurement light LS corresponds to the depth position (z position) of the beam waist of the measurement light LS.

For example, the main controller 211 controls the OCT focusing driver 45A based on a signal-to-noise ratio of the detection result of the interference light LC obtained by OCT measurement or evaluation value(s) (including statistical value of the evaluation values) corresponding to the image quality of the OCT image formed based on the detection result.

When the liquid crystal lens or the Alvarez lens is provided in place of the OCT focusing lens 45, the main controller 211 can control the liquid crystal lens or the Alvarez lens in the same way as it controls the OCT focusing driver 45A.

The main controller 211 controls the light source unit 101. The control for the light source unit 101 includes switching the light source on and off, controlling the intensity of the emitted light, changing the center frequency of the emitted light, changing the sweep speed of the emitted light, changing the sweep frequency, and changing the sweep wavelength range.

The reference driver 114A moves the corner cube 114 provided on the optical path of the reference light along this optical path. Thereby, the difference between the optical path length of the measurement light LS and the optical path length of the reference light LR is changed.

For example, the main controller 211 analyzes the detection result of the interference light LC obtained by OCT measurement (or the OCT image formed based on the detection result), and controls the reference driver 114 A so that the measurement site is positioned at a desired depth position. In some embodiments, any one of the optical path length changing unit 41 and the reference driver 114A is provided.

The main controller 211 controls the polarization controllers 103 and 118. For example, the main controller 211 controls the polarization controllers 103 and 118 based on a signal-to-noise ratio of the detection result of the interference light LC obtained by OCT measurement or evaluation value(s) (including statistical value of the evaluation values) corresponding to the image quality of the OCT image formed based on the detection result.

The main controller 211 controls the attenuator 120. For example, the main controller 211 controls the attenuator 120 based on a signal-to-noise ratio of the detection result of the interference light LC obtained by OCT measurement or evaluation value(s) (including statistical value of the evaluation values) corresponding to the image quality of the OCT image formed based on the detection result.

The main controller 211 controls the detector 125. The control for the detector 125 includes the control for an exposure time (charge accumulation time), a sensitivity, a frame rate, or the like of the detector 125.

The main controller 211 controls the incident polarization control unit 130. Examples of the control for the incident polarization control unit 130 include control of the polarization direction of the incident measurement light LS. Specifically, the main controller 211 can control the incident polarization control unit 130 to switch between a first incident polarization control mode and a second incident polarization control mode. In the first incident polarization control mode, the incident polarization control unit 130 generates the measurement light LS with two polarization states whose polarization directions are orthogonal to each other from the incident measurement light LS, and to emits the generated measurement light LS with two polarization states. In the second incident polarization control mode, the incident polarization control unit 130 generates the measurement light LS with two polarization states superimposed generated as described above, and emits the generated measurement light LS with two polarization states superimposed.

The main controller 211 controls the polarization separation unit 140. Examples of the control for the polarization separation unit 140 include polarization separation control of the returning light of the incident measurement light LS, and emission control of the interference light between the returning light of the measurement light LS, which the polarization separation control has been performed, and the reference light LR. In the polarization separation control, the returning light of the measurement light LS is separated into the returning light of the measurement light LS with two polarization states whose polarization directions are orthogonal to each other. The main controller 211 can control the polarization separation unit 140 to switch between a first emission control mode and a second emission control mode. In the first emission control mode, the interference light with two polarization states is generated by interfering the returning light of the measurement light LS and the reference light LR for each polarization state, and the generated interference light with two polarization states is emitted. In the second emission control mode, the interference light with two polarization states is generated by interfering the returning light of the measurement light LS and the reference light LR for each polarization state, and the generated interference light with two polarization states superimposed is emitted.

The movement mechanism 150 three-dimensionally moves the fundus camera unit 2 (OCT unit 100) relative to the eye E to be examined. For example, the main controller 211 is capable of controlling the movement mechanism 150 to three-dimensionally move the optical system installed in the fundus camera unit 2. This control is used for alignment and/or tracking. Here, the tracking is to move the optical system of the apparatus according to the movement of the eye E to be examined. To perform tracking, alignment and focusing are performed in advance. The tracking is performed by moving the optical system of the apparatus in real time according to the position and orientation of the eye E to be examined based on the image obtained by photographing moving images of the eye E to be examined, thereby maintaining a suitable positional relationship in which alignment and focusing are adjusted.

In some embodiments, the main controller 211 corrects the position of scan range for OCT imaging, based on tracking information obtained by performing tracking (tracking information obtained by tracking the optical system (interference optical system) with respect to a movement of the eye E to be examined). The main controller 211 can control the optical scanner 42 so as to scan the corrected scan range with the measurement light LS.

Such a main controller 211 includes a display controller 211A. The display controller 211A displays the various information on the display apparatus 3 (or display unit 240A described below). Examples of the information displayed on the display apparatus 3 include imaging result (observation image, OCT image), measurement result (measured values), one or more tomographic information images, and data processing result obtained by the data processor 230 for the OCT image or the tomographic information image.

For example, the display controller 211A can display the OCT image and the one or more tomographic information images on the display apparatus 3 or the display unit 240A. Here, in the OCT image, the boundary of the layer region identified by performing segmentation processing has been distinguishably depicted. For example, the display controller 211A can display the OCT image and one of the acquired two or more tomographic information images on the display apparatus 3 or the display unit 240A. Here, also, in the OCT image, the boundary of the layer region identified by performing segmentation processing has been distinguishably depicted. It should be noted that the display controller 211A may display the one or more tomographic information images, in which the boundary of the layer region in the depth direction has been distinguishably depicted, on the display apparatus 3 or the display unit 240A.

Further, the main controller 211 performs a process of writing data in the storage unit 212 and a process of reading out data from the storage unit 212.

### (Storage Unit 212)

The storage unit 212 stores various types of data. Examples of the data stored in the storage unit 212 include detection result(s) of the interference light (scan data), image data of the OCT image and the tomographic information image, image data of the fundus image, and information on the eye to be examined. The information on the eye to be examined includes information on the examinee such as patient ID and name, and information on the eye to be examined such as identification information of the left/right eye.

At least part of the above data stored in the storage unit 212 may be stored in a storage unit provided outside the ophthalmic apparatus 1. For example, the ophthalmic apparatus 1 is connected so as to be capable of communicating with a sever apparatus having a function of storing at least part of the above data via a network such as an in-hospital LAN (Local Area Network). Here, the ophthalmic apparatus 1 and the server apparatus are connected via a WAN (Wide Area Network) such as the Internet. Further, the ophthalmic apparatus 1 and the server apparatus may be connected via a network that combines the LAN and the WAN.

### (Image Forming Unit 220)

An image forming unit 220 forms image data of the OCT image (tomographic image) of the fundus Ef or the anterior segment based on the detection signal (interference signal, scan data) from the detector 125. In other words, the image forming unit 220 generates, as an OCT image generator, the detection result(s) of the interference light with two polarization states superimposed, by synthesizing the detection results of the interference light with two polarization states emitted from the polarization separation unit 140, and forms the image of the eye E to be examined based on the generated detection result(s). This processing includes processes such as noise removal (noise reduction), filter processing, and fast Fourier transform (FFT) in the same manner as the conventional swept source type OCT. The image data acquired in this manner is a data set including a group of image data formed by imaging the reflection intensity profiles of a plurality of A-lines. Here, the A-lines are the paths of the measurement light LS in the eye E to be examined.

In order to improve the image quality, it is possible to repeatedly perform scan with the same pattern a plurality of times to acquire a plurality of data sets, and to compose (i.e., average) the plurality of data sets.

The image forming unit 220 includes, for example, the circuitry described above. It should be noted that “image data” and an “image” based on the image data may not be distinguished from each other in the present specification. In addition, a site of the fundus Ef and an image of the site may not be distinguished from each other.

In some embodiments, the functions of the image forming unit 220 are realized by an image forming processor.

### (Data Processor 230)

Data processor 230 performs various kinds of data processing (e.g., image processing) and various kinds of analysis processing on the detection result of the interference light LC or the image formed by the image forming unit 220. Examples of the data processing include various correction processing such as brightness correction and dispersion correction of the image, and forming processing of the tomographic information image. Examples of the analysis processing include analysis of signal-to-noise ratio of the interference signal, segmentation processing, modification processing of the segmentation processing result, registration processing, and tissue analysis processing in the image.

The tomographic information image is generated using a method different from a method of generating the OCT image and represents tomographic information such as the morphology of the tomographic structure of the eye E to be examined. In the present embodiment, in the generation processing of the tomographic information image, at least one tomographic information image is generated from the detection result(s) of the interference light LC or the OCT image. However, the tomographic information image may be generated without using the detection result(s) of the interference light LC and the OCT image, in the generation processing of the tomographic information image.

Examples of the segmentation processing include identification processing of a plurality of layer regions corresponding to a plurality of layer tissues in the fundus (retina, choroid, etc.) or vitreous body. In the segmentation processing, the boundary of the layer region corresponding to the layer tissue is identified. Examples of the identified layer tissue include a layer tissue that makes up the retina. Examples of the layer tissue that makes up the retina include an inner limiting membrane (ILM), a nerve fiber layer (NFL), a ganglion cell layer (GCL), an inner plexiform layer (IPL), an inner nuclear layer (INL), an outer plexiform layer (OPL), an outer plexiform layer (OPL), an outer nuclear layer (ONL), an external limiting membrane (ELM), a photoreceptor layer, a retinal pigment epithelium (RPE), a choroid, a photoreceptor inner/outersegment junction (IS/OS) or ellipsoid zone (EZ), and a chorio-scleral interface (CSI). In some embodiments, the layer region corresponding to the layer tissue such as a Bruch membrane, a choroid, a sclera or a vitreous body is identified. For example, the layer region corresponding to the layer tissue with a predetermined number of pixels on the sclera side with respect to the RPE is defined as the Bruch membrane.

Examples of the tissue analysis processing in the image include identification processing a predetermined site such as a site of lesion or a tissue, and analysis processing of the composition of a predetermined site. Examples of the site of lesion include a detachment part, a hydrops, a hemorrhage, a lekuma, a tumor, and a drusen. Examples of the tissue include a blood vessel, an optic disc, a fovea, and a macula. Examples of the analysis processing of the composition of the predetermined site include calculation of a distance between designated sites (distance between layers, interlayer distance), an area, an angle, a ratio, or a density; calculation by a designated formula; identification of a shape of a predetermined site; calculation of these statistic values; calculation of distribution of the measured values or the statistic values; image processing based on these analysis processing results.

In some embodiments, the data processor 230 performs the analysis processing on the OCTA image to identify a vessel wall, to identify a vessel region, to identify the connection relationship between two or more vessel regions, to identify the distribution of vessel regions, to identify blood flow, to calculate blood flow velocity, or to determine artery/vein.

Further, the data processor 230 can perform the image processing and/or the analysis processing described above on the image (fundus image, anterior segment image, etc.) obtained by the fundus camera unit 2.

Furthermore, the data processor 230 performs known image processing such as interpolation processing for interpolating pixels between two-dimensional tomographic images to form image data of the three-dimensional image (in the broad sense of the term, OCT image) of the fundus Ef or the eye E to be examined. It should be noted that the image data of the three-dimensional image means image data in which the positions of pixels are defined in a three-dimensional coordinate system. Examples of the image data of the three-dimensional image include image data defined by voxels three-dimensionally arranged. Such image data is referred to as volume data or voxel data. When displaying an image based on volume data, the data processor 230 performs rendering (volume rendering, maximum intensity projection (MIP), etc.) on the volume data, thereby forming image data of a pseudo three-dimensional image viewed from a particular line of sight. The pseudo three-dimensional image is displayed on the display device such as the display unit 240A.

Further, stack data of a plurality of tomographic images may be formed as the image data of the three-dimensional image. The stack data is image data obtained by three-dimensionally arranging tomographic images along a plurality of scan lines based on positional relationship of the scan lines. That is, the stack data is image data obtained by representing tomographic images, which are originally defined in their respective two-dimensional coordinate systems, by a single three-dimensional coordinate system. That is, the stack data is image data formed by embedding tomographic images into a single three-dimensional space.

The data processor 230 can perform position matching between the fundus image and the OCT image. When the fundus image and the OCT image are obtained in parallel, the position matching between the fundus image and the OCT image, which have been (almost) simultaneously obtained, can be performed using the optical axis of the imaging optical system 30 as a reference. Such position matching can be achieved since the optical system for the fundus image and that for the OCT image are coaxial. Besides, regardless of the timing of obtaining the fundus image and the OCT image, position matching between the fundus image and the OCT image can be achieved by registering the fundus image with an image obtained by projecting the OCT image onto the x-y plane. This position matching method can also be employed when the optical system for obtaining the fundus image and the optical system for OCT measurement are not coaxial. Further, when both the optical systems are not coaxial, if the relative positional relationship between these optical systems is known, the position matching can be performed with referring to the relative positional relationship in a manner similar to the case of coaxial optical systems.

As shown in FIG. 4, the data processor 230 includes a tomographic information image generator 231, a segmentation processor 232, and a modification processor 233.

The tomographic information image generator 231 is configured to generate the tomographic information image from the detection result(s) of the interference light LC or the OCT image. The tomographic information image is a tomographic image generated using a method different from a method of generating the OCT image. Here, the OCT image may be an OCT image formed by the image forming unit 220, or an OCT image obtained by performing data processing such as brightness correction on the OCT data, which is formed by the image forming unit 220, by the data processor 230.

The segmentation processor 232 is configured to perform known segmentation processing on the OCT image to divide the layer regions that make up the tomographic structure in the depth direction, and to perform processing for identifying the boundaries of the layer regions. Here, also, the OCT image may be an OCT image formed by the image forming unit 220, or an OCT image formed by the image forming unit 220, on which data processing such as brightness correction is performed by the data processor 230. In some embodiments, the segmentation processor 232 is configured to perform the segmentation processing on the tomographic information image.

The modification processor 233 is configured to perform processing for modifying the boundary of the layer region identified by performing segmentation processing, based on the operation information input by the user via the operation unit 240B described below, while referring to the tomographic information image generated by the tomographic information image generator 231.

### (Tomographic Information Image Generator 231)

The tomographic information image generator 231 includes an OCTA image generator 231A, an attenuation coefficient image generator 231B, a DOPU image generator 231C, and a birefringence image generator 231D.

### (OCTA Image Generator 231A)

The OCTA image generator 231A generates the OCTA image based on the detection result(s) of the interference light or the OCT image formed based on the detection result(s) of the interference light. The OCTA image is a motion contrast image representing the distribution of the contrast intensity that varies due to motion at each pixel position. The OCTA image is an angiogram or a vascular enhancement image in which the retinal blood vessels and/or the choroid blood vessels are emphasized. In the OCTA image representing the tomographic information on the fundus Ef, the boundaries of the ILM, the INL, the OPL, and the RPE are especially highlighted compared to the OCT image (tomographic image) formed by the image forming unit 220.

The OCTA image generator 231A generates the OCTA image as the motion contrast image by repeatedly performing OCT scans on (almost) the same cross-section surface in the eye E to be examined. In other words, the OCTA image generator 231A generates the OCTA image based on the scan data acquired chronologically by performing OCT scans on almost the same scan position in the eye E to be examined.

For example, the OCTA image generator 231A compares two OCT images or scan data acquired by repeatedly performing OCT scans on almost the same site in the eye E to be examined. The OCTA image generator 231A converts the pixel values of the changed parts of the signal intensity by comparing the two OCT images or scan data into pixel values corresponding to the amount of the change, and generates the OCTA image in which the parts that have changed are emphasized.

In some embodiments, the OCTA image generator 231A can extract information for a predetermined thickness at a desired site from a plurality of generated OCTA images to build an image as an en-face image.

### (Attenuation Coefficient Image Generator 231B)

The attenuation coefficient image generator 231B generates the attenuation coefficient image based on the detection result(s) of the interference light or the OCT image formed based on the detection result(s) of the interference light. The power of the measurement light LS as coherent light is attenuated by scattering and absorption during propagation through the medium. The attenuation coefficient image is an image representing the distribution of the attenuation coefficient of the irradiance of the measurement light LS, which depends on the optical characteristics of the medium, as the distribution of the irradiance of the measurement light LS. Examples of the attenuation coefficient include an attenuation coefficient when representing an irradiance that attenuates in the depth direction according to Lambert-Beer's Law for the irradiance of incident light (ray) at a reference position in the depth direction. Such a distribution of the attenuation coefficients may be useful in acquiring information on the composition of the medium. In the attenuation coefficient image representing the tomographic information on the fundus Ef, the boundaries of the ILM, the EZ, the RPE, and the CSI are depicted with particular emphasis compared to the OCT image (tomographic image) formed by the image forming unit 220.

The attenuation coefficient image generator 231B, for example, generates the attenuation coefficient image by replacing the pixel values (brightness values) at each pixel position in the OCT image with pixel values corresponding to the attenuation coefficient generated based on the pixel values in the OCT image.

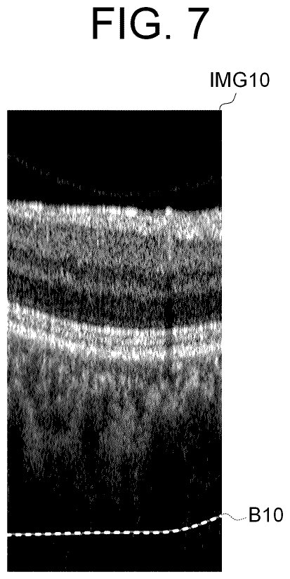

FIG. 6 shows a diagram for explaining the operation of the attenuation coefficient image generator 231B. FIG. 6 schematically represents the operation of the attenuation coefficient image generator 231B when calculating the pixel value of the pixel P1 in the attenuation coefficient image IMG1 corresponding to the pixel P in the OCT image IMG0.

Assuming that the pixel of interest in the OCT image IMG0 is the pixel P, the attenuation coefficient image generator 231B first identifies the pixel values of one or more pixels in the A-scan direction (depth direction) that pass through the pixel P for the OCT image IMG0. Next, the attenuation coefficient image generator 231B obtains the pixel value of the pixel P1 in the attenuation coefficient image IMG1 as the value obtained by dividing the pixel value of the pixel P by the cumulative sum of the pixel values of one or more pixels located deeper than the pixel P in the OCT image IMG0.

For example, the attenuation coefficient image generator 231B obtains the pixel value Ia(i) of the pixel P1 at depth position “i” in the attenuation coefficient image IMG1 corresponding to the pixel P at the depth position “i” in the OCT image IMG0 according to Equation (1), as described in “Depth-resolved model-based reconstruction of attenuation coefficients in optical coherence tomography” (K. A. Vermeer et. al, Jan. 1, 2014, Vol. 5, No. 1, DOI: 10.1364/BOE.5.000322, BIOMEDICAL OPTICS EXPRESS, pp. 322-337).

$$\begin{matrix}[ Equation 1 ] & \\ Ia ( i ) = \frac{1}{2 Δ} log ( 1 + \frac{I [ i ]}{{\sum }_{i + 1}^{∞} I [ i ]} ) & ( 1 )\end{matrix}$$

In Equation (1), “Δ” represents the pixel size in the depth direction, “i” represents the depth position, and “I[i]” represents the pixel value (brightness value) at depth position “i” in OCT image IMG0.

In some embodiments, the attenuation coefficient image generator 231B performs correction that takes into account light absorption, multiple scattering, and diffusion on the pixel value Ia (i) obtained by Equation (1).

The attenuation coefficient image generator 231B generates the attenuation coefficient image IMG1 by repeating the above processing for each pixel in the OCT image IMG0.

### (DOPU Image Generator 231C)

The DOPU image generator 231C generates the DOPU image based on at least the detection result(s) of the interference light obtained by emitting the interference light with two polarization states, which is synthesized for each polarization state, from the polarization separation unit 140, as described above. The DOPU image is an image representing the distribution of the uniformity of polarization of the measurement light LS propagating through the medium. In the DOPU image representing the tomographic information on the fundus Ef, the boundaries of the RPE, the choroid, and the CSI are depicted with particular emphasis compared to the OCT image (tomographic image) formed by the image forming unit 220.

For example, the DOPU image generator 231C generates the DOPU image by obtaining the pixel value of each pixel in the DOPU image based on the detection result(s) of the interference light detected for each polarization state, as described in “Degree of polarization uniformity with high noise immunity using polarization-sensitive optical coherence tomography” (S. Makita et. al. coherence tomography” (S. Makita et. al, Dec. 15, 2014, Vol. 39, No. 24, OPTICS LETTERS, pp. 6783-6786).

In some embodiments, the DOPU image generator 231C generates the DOPU image by obtaining the pixel value of each pixel in the DOPU image using the pixel values of the OCT image formed for each polarization state by the image forming unit 220.

### (Birefringence Image Generator 231D)

The birefringence image generator 231D generates the birefringence image, as described above, based on the detection result(s) of interference light obtained by emitting measurement light LS with two polarization states superimposed from the incident polarization control unit 130 and emitting the interference light with two polarization states synthesized for each polarization state from the polarization separation unit 140. The birefringence image is an image representing the distribution of the birefringence of the measurement light propagating through the medium. In the birefringence image representing the tomographic information on the fundus Ef, the boundaries of the ILM and the RPE are depicted with particular emphasis compared to the OCT image (tomographic image) formed by the image forming unit 220.

For example, the birefringence image generator 231D generates the birefringence image by obtaining the pixel value of each pixel in the birefringence image based on the interference light detected for each polarization state, as described in “Birefringence imaging of posterior eye by multi-functional Jones matrix optical coherence tomography” (S. Sugiyama et. al, Dec. 1, 2015, Vol. 6, No. 12, DOI: 10.1364/BOE.6.004951, BIOMEDICAL OPTICS EXPRESS, pp. 4951-4974)”.

In some embodiments, the birefringence image generator 231D generates the birefringence image by obtaining the pixel value at each pixel in the birefringence image using the pixel values of the OCT image formed for each polarization state by the image forming unit 220.

In addition, the tomographic information image generator 231 can generate a superimposed image obtained by superimposing any one more of the OCTA image, the attenuation coefficient image, the DOPU image, and the birefringence image. When any one of the OCTA image, the attenuation coefficient image, the DOPU image, and the birefringence image has been selected as a reference image, the superimposed image is an image in which one or more of the OCTA image, the attenuation coefficient image, the DOPU image, and the birefringence image, excluding the above reference image, are superimposed on the reference image.

### (Modification Processor 233)

The modification processor 233 perform modification processing for replacing the boundary of the layer region in the OCT image identified by the segmentation processor 232 with a modified boundary. The modified boundary is set based on the operation information input (entered) by the user, such as a doctor, via the operation unit 240B. In some embodiments, the modified boundary is a boundary of the layer region identified by the segmentation processing on the tomographic information image.

The data processor 230 that functions described above includes, for example, a processor described above, a RAM, a ROM, a hard disk drive, a circuit board, and the like. Computer programs that cause a processor to execute the above functions are previously stored in a storage device such as a hard disk drive. In some embodiments, the functions of data processor 230 are realized by one or more data processors.

### (User Interface 240)

As shown in FIG. 3, the user interface 240 includes the display unit 240A and the operation unit 240B. The display unit 240A includes the display device of the arithmetic control unit 200 described above and/or the display apparatus 3. The operation unit 240B includes the operation device of the arithmetic control unit 200 described above. The operation unit 240B may include various kinds of buttons and keys provided on the housing of the ophthalmic apparatus 1, or provided outside the ophthalmic apparatus 1. For example, when the fundus camera unit 2 has a case similar to that of the conventional fundus camera, the operation unit 240B may include a joy stick, an operation panel, and the like provided to the case. Further, the display unit 240A may include various kinds of display devices, such as a touch panel placed on the housing of the fundus camera unit 2.

It should be noted that the display unit 240A and the operation unit 240B need not necessarily be formed as separate devices. For example, a device like a touch panel, which has a display function integrated with an operation function, can be used. In such cases, the operation unit 240B includes the touch panel and a computer program. The content of operation performed on the operation unit 240B is fed to the controller 210 as an electric signal. Moreover, operations and inputs of information may be performed using a graphical user interface (GUI) displayed on the display unit 240A and the operation unit 240B.

The data processor 230 (and the image forming unit 220) is/are an example of the “ophthalmic information processing apparatus” according to the embodiments. The optical system included in the OCT unit 100, the image forming unit 220, and the tomographic information image generator 231, or the communication unit (not shown) are an example of the “acquisition unit” according to the embodiments. The optical system included in the OCT unit 100 is an example of the “optical system” according to the embodiments. The display apparatus 3 or the display unit 240A is an example of the “display means” according to the embodiments. The OCTA image, the DOPU image, and the birefringence image are examples of the “tomographic information image” according to the embodiments. The DOPU image is an example of the “polarization information image” according to the embodiments.

The segmentation processing performed by the segmentation processor 232 generally depends on the image quality of the image to be processed, which often makes it difficult to identify the boundary of the layer region with high accuracy. Therefore, various methods have been proposed to improve the accuracy of segmentation processing results. However, the extent of the proposed methods to improve accuracy in specific diseases or specific cases is limited. Thus, at present, a user such as a doctor needs to check the boundary of the layer region obtained by the segmentation processing. And, if necessary, the user needs to modify the boundary of the layer region.

Hereinafter, a case where the boundary of the CSI identified by the segmentation processing in the OCT image, using the attenuation coefficient image is modified, will be described. However, the following description can be applied to other tomographic information images other than attenuation coefficient images, in the same way. Further, the following description can be applied to the boundaries of other layer regions other than the boundary of the CSI, in the same way.

FIG. 7 shows an example of the processing result of the segmentation processing performed by the segmentation processor 232 according to the embodiments. FIG. 7 represents an example of the CSI identified by the segmentation processing for the OCT image IMG10, which is a tomographic image formed by the image forming unit 220.

As shown in FIG. 7, the CSI (boundary B10) identified by the segmentation processing performed by the segmentation processor 232 can be found to be different from the actual CSI if a doctor or other person checks it. Therefore, in the present embodiment, focusing on the fact that different layer regions (or their boundaries) are distinctly depicted in the tomographic information image from those in the OCT image, the OCT image IMG10 and the one or more tomographic information images are displayed on the display unit 240A.

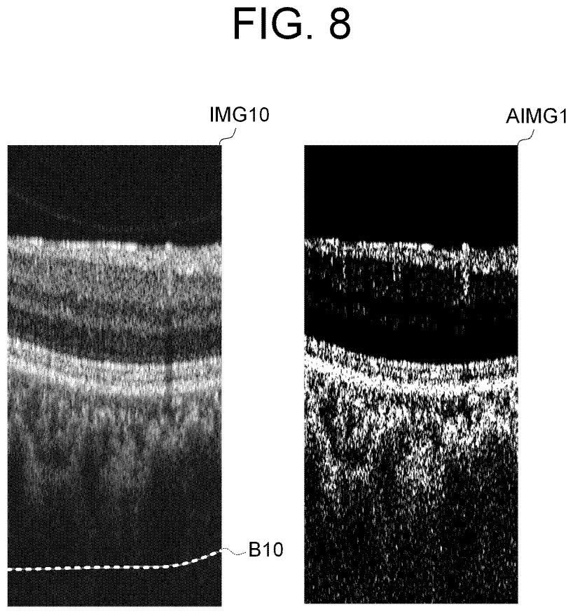

FIG. 8 schematically shows an example of the parallel display of the OCT image IMG10 in FIG. 7 and the attenuation coefficient image AIMG1 on the same screen of the display unit 240A.

As shown in FIG. 8, the boundary of the CSI (the true boundary, which is different from the boundary B10) is depicted indistinctly in the OCT image IMG10. However, the boundary of the CSI is more distinctly depicted in the attenuation coefficient image AIMG1. Therefore, the user can modify the position of the boundary B10 of the CSI in the OCT image IMG10 by inputting the operation information via the operation unit 240B while referring to the boundary of the CSI depicted in the attenuation coefficient image AIMG1.

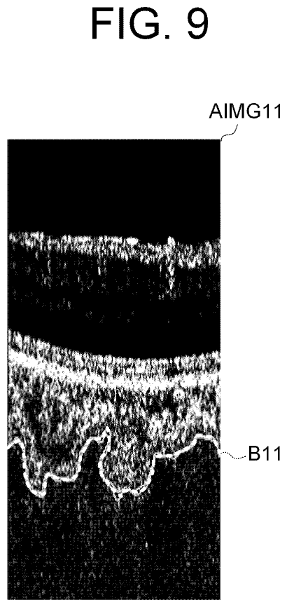

FIG. 9 schematically shows the boundary of the CSI in the attenuation coefficient image in FIG. 8. In FIG. 9, the boundary B11 of the CSI in the attenuation coefficient image AIMG11 is represented.

For example, the user inputs operation information via the operation unit 240B while referring to the attenuation coefficient image AIMG1, and modifies the boundary B10 of the CSI in the OCT image IMG10. The modification processor 233 sets the boundary of the CSI modified based on the operation information as the boundary of the CSI in the OCT image IMG10.

For example, the user inputs the operation information via the operation unit 240B while referring to the attenuation coefficient image AIMG1, and designates the boundary of the CSI on the OCT image IMG10. The modification processor 233 sets the boundary defined by the position designated on the OCT image IMG10 based on the operation information as the boundary in the OCT image IMG10 instead of the boundary B10 of the CSI.

In some embodiments, the display controller 211A displays the OCT image IMG10 and the attenuation coefficient image AIMG1 in a superimposed manner. The user traces the boundary of the CSI depicted in the attenuation coefficient image AIMG1, which is superimposed on the OCT image IMG10. The modification processor 233 sets the boundary defined by the position designated on the attenuation coefficient image AIMG1 based on the operation information as the boundary in the OCT image IMG10 instead of the boundary B10 of the CSI.

In addition, since the registration processing between the OCT image IMG10 and the attenuation coefficient image AIMG1 is unnecessary, as described above, the pixel positions of the OCT image IMG10 correspond the pixel positions of the attenuation coefficient image AIMG1 one-to-one. In other words, the pixel in the OCT image IMG10 corresponding to the pixel of interest in the attenuation coefficient image AIMG1 can be easily identified. In some embodiments, the user traces the boundary of the CSI depicted in the attenuation coefficient image AIMG1 without displaying the OCT image IMG10 and the attenuation coefficient image AIMG1 in a superimposed manner. The modification processor 233 identifies the position on the OCT image IMG10 corresponding to the position of the boundary B11 of the CSI defined by the position designated on the attenuation coefficient image AIMG1 based on the operation information, and sets the boundary of the identified position as the boundary in the OCT image IMG10 instead of the boundary B10 of the CSI.

Alternatively, without displaying the OCT image IMG10 and the attenuation coefficient image AIMG1 in parallel (e.g., displaying only the attenuation coefficient image AIMG1), the user traces the boundary of the CSI depicted in the attenuation coefficient image AIMG1. The modification processor 233 identifies the position on the OCT image IMG10 corresponding to the position of the boundary B11 of the CSI defined by the position designated on the attenuation coefficient image AIMG1 based on the operation information, and sets the boundary of the identified position as the boundary in the OCT image IMG10 instead of the boundary B10 of the CSI.

In some embodiments, every time the position of the boundary of the CSI is designated based on the operation information for the attenuation coefficient image AIMG1, the corresponding position(s) in the OCT image IMG10 is/are identified and the identified corresponding position(s) is/are distinguishably displayed in real time.

In some embodiments, the segmentation processor 232 performs the segmentation processing on the attenuation coefficient image AIMG1 to identify the boundary B11 of the CSI. The modification processor 233 sets the boundary B11 of the CSI defined by the segmentation processor 232 as the boundary in the OCT image IMG10 instead of the boundary B10 of the CSI.

In FIG. 8 and FIG. 9, the case where the OCT image and a single tomographic information image other than the OCT image are displayed on display unit 240A has been described. However, the configuration according to the embodiments is not limited to this.

For example, the OCT image and two or more tomographic information images other than the OCT image may be displayed on the display unit 240A to modify the layer region in the OCT image.

For example, the OCT image and one or more tomographic information images other than the OCT image may be displayed on the display unit 240A, and the boundaries of a plurality of the layer regions identified by the segmentation processing in the OCT image may be modified using the one or more tomographic information images. In other words, the one or more tomographic information images include a first tomographic information image and a second tomographic information image different from the first tomographic information image. The display controller 211A is configured to display the OCT image, in which a first layer region is depicted, and the first tomographic information image, on the display unit 240A, and to display the OCT image, in which a second layer region different from the first layer region is depicted, and the second tomographic information image, on the display unit 240A.

Furthermore, the segmentation result for the OCT image and the segmentation result for the attenuation coefficient image may be superimposed on the OCT image.

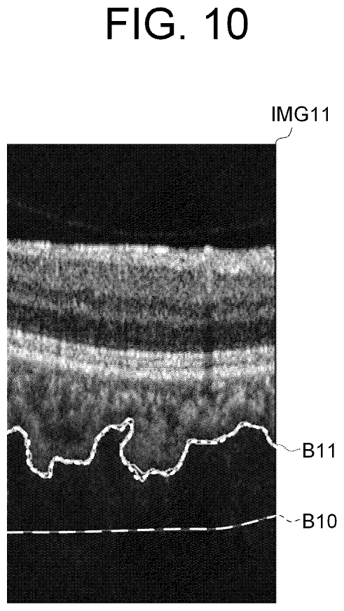

FIG. 10 schematically shows an example of the superimposed display of the segmentation result for the OCT image and the segmentation result for the attenuation coefficient image on the OCT image.

The segmentation processor 232 identifies the boundary B10 of the CSI by performing the segmentation processing on the OCT image IMG1, and identifies the boundary B11 of the CSI by performing the segmentation processing on the attenuation coefficient image AIMG1. The display controller 211A displays the OCT image IMG11 on the display unit 240A. Here, The OCT image IMG11 is an image in which the boundary B10 and the boundary B11 are superimposed on the OCT image IMG1. In this case, the attenuation coefficient image AIMG1 may be superimposed on the OCT image IMG1.

The user designates the boundary B10 or the boundary B11 on the OCT image IMG11 by inputting the operation information via the operation unit 240B. The modification processor 233 sets the boundary B10 or the boundary B11 designated by the user as the boundary in the OCT image IMG10.

### Operation Example

The operation of the ophthalmic apparatus 1 according to the embodiments will be described. Hereinafter, it is assumed that the incident polarization control unit 130 is set to the first incident polarization control mode and the polarization separation unit 140 is set to the first emission control mode.

### First Operation Example

In the first operation example, the boundary of the layer region identified by performing the segmentation processing on the OCT image is manually modified while referring to the one or more tomographic information images.

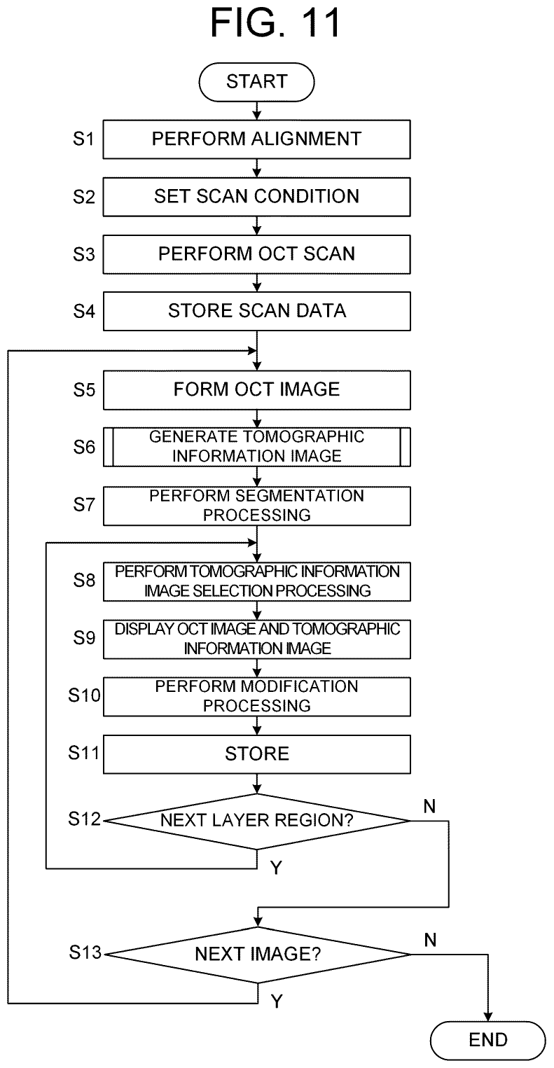

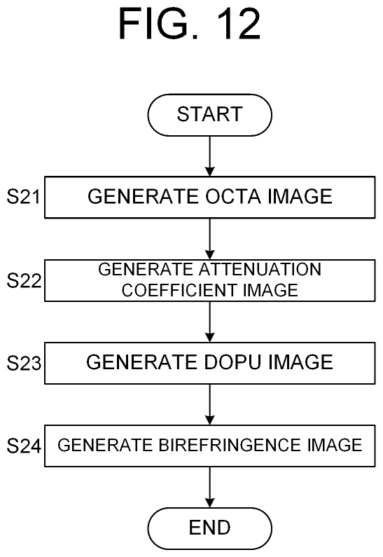

FIG. 11 and FIG. 12 show flow diagrams of the first operation example of the ophthalmic apparatus 1 according to the embodiments. The storage unit 212 stores computer program(s) for realizing the processing shown in FIG. 11 and FIG. 12. The main controller 211 operates according to the computer program(s), and thereby the main controller 211 performs the processing shown in FIG. 11 and FIG. 12.

### (S1: Perform Alignment)

First, the main controller 211 performs alignment adjustment of the optical system relative to the eye E to be examined in a state where the fixation target is presented at a predetermined fixation position. Examples of the alignment adjustment include manual alignment and automatic alignment.

When the alignment adjustment is performed manually, the main controller 211 controls the alignment optical system 50 to project a pair of alignment indicators onto the eye E to be examined. A pair of alignment bright spots are displayed on the display unit 240A as the light receiving images of these alignment indicators. Further, the main controller 211 displays an alignment scale representing the target position of movement of the pair of alignment bright spots on the display unit 240A. The alignment scale is, for example, a bracket type image.

When the positional relationship between the eye E to be examined and the fundus camera unit 2 (objective lens 22) is appropriate, the pair of alignment bright spots are once imaged at a predetermined position (for example, intermediate position between the corneal apex and the center of corneal curvature) respectively, and is projected onto the eye E to be examined, according to a known method. In this case, the case where the positional relationship described above is appropriate is the case where the distance (working distance) between the eye E to be examined and the fundus camera unit 2 is appropriate and the optical axis of the optical system of the fundus camera unit 2 and the ocular axis (corneal apex position) of the eye E to be examined are (approximately) coincident. The examiner (user) can perform the alignment adjustment of the optical system to the eye E to be examined by moving the fundus camera unit 2 three-dimensionally so as to guide the pair of alignment bright spots into the alignment scale.

When the alignment adjustment is performed automatically, the movement mechanism 150 for moving the fundus camera unit 2 is used. The data processor 230 identifies the position of each alignment bright spot in the screen displayed on display unit 240A, and obtains a displacement between the identified position of each alignment bright point and the alignment scale. The main controller 211 controls the movement mechanism 150 to move the fundus camera unit 2 so as to cancel this displacement. Identifying the position of each alignment bright spot can be performed, for example, by obtaining the brightness distribution of each alignment bright spot and obtaining the position of the center of gravity based on this brightness distribution. Since the position of the alignment scale is constant, the desired displacement can be obtained, for example, by calculating the displacement between the center position of the alignment scale and the above position of the center of gravity. The movement direction and the movement distance of the fundus camera unit 2 can be determined by referring to a preset unit movement distances in the x direction, y direction, and z direction (e.g., the result of prior measurement of how much the alignment indicator moves in which direction, when the fundus camera unit 2 is moved by how much in which direction). The main controller 211 generates signals according to the determined movement direction and movement distance, and transmits these signals to the movement mechanism 150. Thereby, the position of the optical system relative to the eye E to be examined is changed automatically.

### (S2: Set Scan Condition)

Next, the main controller 211 sets the scan condition(s) so as to scan a desired scan region with a desired scan mode.

For example, the user designates the scan position (scan region) for the OCT scan on the fundus image (front image) of the eye E to be examined previously acquired using the fundus camera unit 2 (imaging optical system 30) by inputting (entering) the operation information via the operation unit 240B. As described above, the OCT scan can be easily performed on the scan position (scan region) designated on the fundus image because the registration between the fundus image and the OCT image is unnecessary.

### (S3: Perform OCT Scan)

Subsequently, the main controller 211 controls the optical scanner 42, the OCT unit 100, and the like to perform OCT scan under the scan condition set in step S2. The main controller 211 can repeatedly perform OCT scan on the same scan position to acquire the OCTA image.

### (S4: Store Scan Data)

The main controller 211 stores the scan data obtained in step S3 in the storage unit 212. The scan data stored in step S4 is three-dimensional scan data.

### (S5: Form OCT Image)

Subsequently, the main controller 211 controls the image forming unit 220 to form a single OCT image (B-scan image), which is a tomographic image at a predetermined slice position, from the scan data stored in step S4.

### (S6: Generate Tomographic Information Image)

Subsequently, the main controller 211 controls the tomographic information image generator 231 to generate the one or more tomographic information images at the same slice position as the OCT image formed in step S5, based on the scan data stored in step S4 or the OCT image formed in step S5. In the present embodiment, the OCTA image, the DOPU image, and the birefringence image are generated in step S6.

Details of step S6 will be described later.

### (S7: Perform Segmentation Processing)

Next, the main controller 211 controls the segmentation processor 232 to perform the segmentation processing on the OCT image formed in step S5 to identify the boundary of a desired layer region.

It should be noted that the order of performing steps S6 and S7 may be interchanged.

### (S8: Perform Tomographic Information Image Selection Processing)

Next, the main controller 211 causes the selection processing of the tomographic information image generated in step S6 to be performed. The user selects at least one of the one or more tomographic information images formed in step S6 using the operation unit 240B.

### (S9: Display OCT Image and Tomographic Information Image)

Subsequently, the main controller 211 controls the display controller 211A to display the OCT image, in which the boundary of the desired layer region identified in step S7 is distinguishably depicted, and the one or more tomographic information images selected in step S8 on the display unit 240A.

### (S10: Perform Modification Processing)

Subsequently, the main controller 211 controls the modification processor 233 to perform the modification processing for modifying the boundary of the layer region identified in step S7 in the OCT image.

For example, the user inputs the operation information via operation unit 240B while referring to the one or more tomographic information images displayed on the display unit 240A in step S9, and modifies the boundary of the layer region in the OCT image. The modification processor 233 sets the boundary of the layer region modified based on the operation information as the boundary of the layer region identified in step S7 in the OCT image.

### (S11: Store)

Subsequently, the main controller 211 stores the OCT image, in which the boundary of the layer region has been modified in the modification processing in step S10, in the storage unit 212.

### (S12: Next Layer Region?)

Subsequently, the main controller 211 determines whether or not there is a boundary of a layer region to be modified next. For example, the main controller 211 determines whether or not there is a boundary of a layer region to be modified next, by determining whether or not the boundaries of the layer regions previously determined to be modified have been completed.

When it is determined in step S12 that there is a boundary of a layer region to be modified next (S12: Y), the operation of the ophthalmic apparatus 1 proceeds to step S8. When it is determined in step S12 that there is not a boundary of a layer region to be modified next (S12: N), the operation of the ophthalmic apparatus 1 proceeds to step S13.

### (S13: Next Image?)

When it is determined in step S12 that there is not a boundary of a layer region to be modified next (S12: N), the main controller 211 determines whether or not there is an image in which a boundary of a layer region should be modified next. For example, the main controller 211 determines whether or not there is an image in which a boundary of a layer region should be modified next, by determining whether or not the modification processing has been completed for the OCT image with a predetermined number of slices.

When it is determined in step S13 that there is an image in which a boundary of a layer region should be modified next (S13: Y), the operation of the ophthalmic apparatus 1 proceeds to step S5. After proceeding to step S5, steps S5 through S13 are performed sequentially for the OCT images at the next slice position. When it is determined in step S13 that there is not an image in which a boundary of a next region should be modified next (S13: N), the operation of the ophthalmic apparatus 1 is terminated (END).

Step S6 in FIG. 11 is processed according to the flow shown in FIG. 12.

### (S21: Generate OCTA Image)

In step S6, the main controller 211 controls the OCTA image generator 231A to generate the OCTA image from the chronological scan data stored in step S4 (or the chronological OCT images formed in step S5).

The OCTA image generator 231A generates the OCTA image as described above.

### (S22: Generate Attenuation Coefficient Image)

Subsequently, the main controller 211 controls the attenuation coefficient image generator 231B to generate the attenuation coefficient image from the OCT image formed in step S5.

The attenuation coefficient image generator 231B generates the attenuation coefficient image as described above.

### (S23: Generate DOPU Image)

Subsequently, the main controller 211 controls the DOPU image generator 231C to generate the DOPU image based on the scan data stored in step S4.

The DOPU image generator 231C generates the DOPU image as described above.

### (S24: Generate Birefringence Image)

Subsequently, the main controller 211 controls the birefringence image generator 231D to generate the birefringence image based on the scan data stored in step S4.

The birefringence image generator 231D generates the birefringence image as described above.

It should be noted that the order of execution of steps S21 through S24 can be changed as desired.

This terminates the processing of step S6 in FIG. 11 (END).

### Second Operation Example

In the second operation example, the boundary of the layer region in the OCT image is manually modified from the boundary of the layer region identified by performing the segmentation processing on the OCT image and the boundary of the layer region identified by performing the segmentation process on each of the one or more tomographic information images.

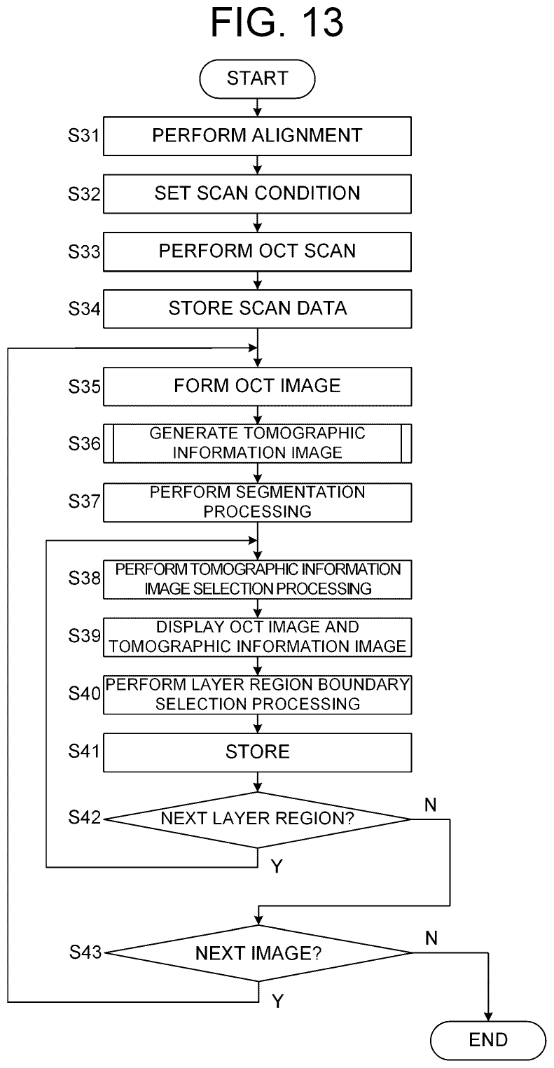

FIG. 13 shows a flow diagram of the second operation example of the ophthalmic apparatus 1 according to the embodiments. The storage unit 212 stores computer program(s) for realizing the processing shown in FIG. 13 and FIG. 12. The main controller 211 operates according to the computer program(s), and thereby the main controller 211 performs the processing shown in FIG. 13 and FIG. 12.

### (S31: Perform Alignment)

First, the main controller 211 performs alignment adjustment of the optical system relative to the eye E to be examined, in the same manner as in step S1.

### (S32: Set Scan Condition)

Next, the main controller 211 sets the scan condition(s) so as to scan a desired scan region with a desired scan mode, in the same manner as in step S2.

### (S33: Perform OCT Scan)

Subsequently, the main controller 211 controls the optical scanner 42, the OCT unit 100, and the like to perform OCT scan under the scan condition set in step S32, in the same manner as in step S3.

### (S34: Store Scan Data)

The main controller 211 stores the scan data obtained in step S33 in the storage unit 212, in the same manner as in step S4.

### (S35: Form OCT Image)

Subsequently, the main controller 211 controls the image forming unit 220 to form a single OCT image (B-scan image), which is a tomographic image at a predetermined slice position, from the scan data stored in step S34, in the same manner as in step S5.

### (S36: Generate Tomographic Information Image)

Subsequently, the main controller 211 controls the tomographic information image generator 231 to generate the one or more tomographic information images at the same slice position as the OCT image formed in step S35, based on the scan data stored in step S34 or the OCT image formed in step S35, in the same manner as in step S6.

### (S37: Perform Segmentation Processing)

Next, the main controller 211 controls the segmentation processor 232 to perform the segmentation processing on the OCT image formed in step S35 and each of the one or more tomographic information images generated in step S36 to identify the boundaries of a desired layer region.

### (S38: Perform Tomographic Information Image Selection Processing)

Next, the main controller 211 causes the selection processing of the tomographic information image generated in step S36 to be performed, in the same manner as in step S8. The user selects at least one of the one or more tomographic information images formed in step S36 using the operation unit 240B.

### (S39: Display OCT Image and Tomographic Information Image)

Subsequently, the main controller 211 controls the display controller 211A to display the OCT image, in which the boundary of the desired layer region identified in step S37 is distinguishably depicted, and the one or more tomographic information images, in which the boundary of the desired layer region is distinguishably depicted, selected in step S38 on the display unit 240A, in the same manner as in step S9.

### (S40: Perform Layer Region Boundary Selection Processing)

Subsequently, the main controller 211 controls the modification processor 233 to select any one of the following displayed in step S39: the boundary of the desired layer region identified in the OCT image, or the boundary of the desired layer region selected in step S38 and identified in the tomographic information image.

For example, the user inputs the operation information via the operation unit 240B and selects any one of the OCT image in which the boundary of the desired layer region is distinguishably depicted, and the one or more tomographic information images in which the boundary of the desired layer region is distinguishably depicted, which are displayed on the display unit 240A in step S39. The modification processor 233 sets the boundary of the layer region in any of the OCT image or the one or more tomographic information images selected based on the operation information as the boundary of the layer region in the OCT image identified in step S37.

### (S41: Store)

Subsequently, the main controller 211 stores the OCT image in which the boundary of the layer region has been modified in the layer region boundary selection processing in step S40 in the storage unit 212.

### (S42: Next Layer Region?)

Subsequently, the main controller 211 determines whether or not there is a boundary of a layer region to be modified next, in the same manner as in step S12.

When it is determined in step S42 that there is a boundary of a layer region to be modified next (S42: Y), the operation of the ophthalmic apparatus 1 proceeds to step S38. When it is determined in step S42 that there is not a boundary of a layer region to be modified next (S42: N), the operation of the ophthalmic apparatus 1 proceeds to step S43.

### (S43: Next Image?)

When it is determined in step S42 that there is not a boundary of a layer region to be modified next (S42: N), the main controller 211 determines whether or not there is an image in which a boundary of a layer region should be modified next, in the same manner as in step S13.

When it is determined in step S43 that there is an image in which a boundary of a layer region should be modified next (S43: Y), the operation of the ophthalmic apparatus 1 proceeds to step S35. After proceeding to step S35, steps S35 through S43 are performed sequentially for the OCT images at the next slice position. When it is determined in step S43 that there is not an image in which a boundary of a next region should be modified next (S43: N), the operation of the ophthalmic apparatus 1 is terminated (END).

### (Third Operation Example)

In the third operation example, the boundary of the layer region identified by performing the segmentation processing on the OCT image is manually modified while referring to the one or more tomographic information images acquired from outside the ophthalmic apparatus 1. In this case, it is assumed that the ophthalmic apparatus 1 is capable of communicating with an external ophthalmic apparatus capable of acquiring the one or more tomographic information images via a network, or a server apparatus that stores electronic medical record information in which the one or more tomographic information images are associated with the eye to be examined.

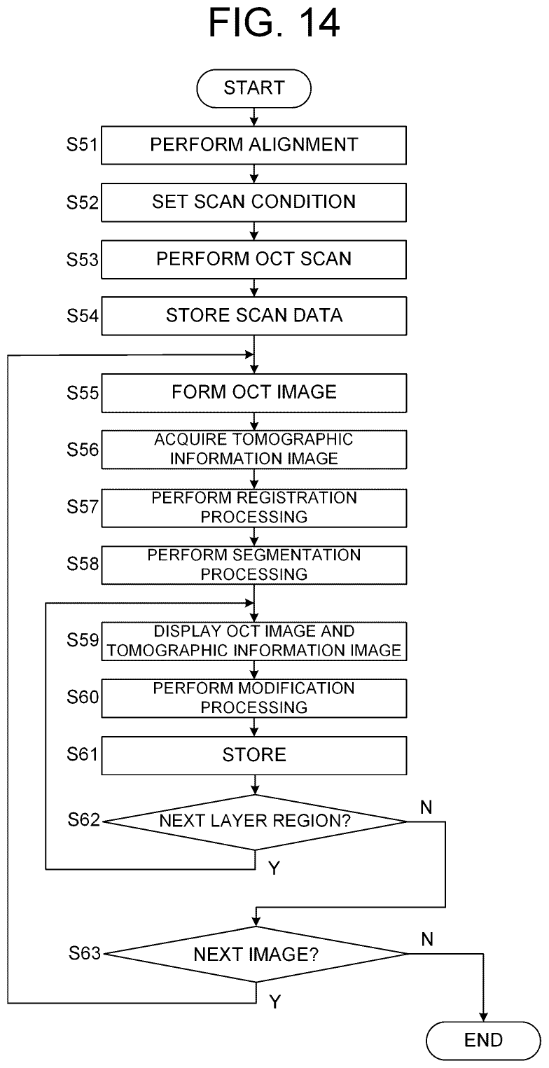

FIG. 14 shows a flow diagram of the third operation example of the ophthalmic apparatus 1 according to the embodiments. The storage unit 212 stores a computer program for realizing the processing shown in FIG. 14. The main controller 211 operates according to the computer program(s), and thereby the main controller 211 performs the processing shown in FIG. 14.

### (S51: Perform Alignment)

First, the main controller 211 performs alignment adjustment of the optical system relative to the eye E to be examined, in the same manner as in step S1.

### (S52: Set Scan Condition)

Next, the main controller 211 sets the scan condition(s) so as to scan a desired scan region with a desired scan mode, in the same manner as in step S2.

### (S53: Perform OCT Scan)

Subsequently, the main controller 211 controls the optical scanner 42, the OCT unit 100, and the like to perform OCT scan under the scan condition set in step S52, in the same manner as in step S3.

### (S54: Store Scan Data)

The main controller 211 stores the scan data obtained in step S53 in the storage unit 212, in the same manner as in step S4.

### (S55: Form OCT Image)

Subsequently, the main controller 211 controls the image forming unit 220 to form a single OCT image (B-scan image), which is a tomographic image at a predetermined slice position, from the scan data stored in step S54, in the same manner as in step S5.

### (S56: Acquire Tomographic Information Image)

Subsequently, the main controller 211 controls a communication unit (not shown) to acquire the one or more tomographic information images at (almost) the same slice position as the OCT image formed in step S55 from the ophthalmic apparatus or the server apparatus, the ophthalmic apparatus or the server apparatus being provided outside the ophthalmic apparatus 1.

For example, the main controller 211 can control the communication unit to send the slice position(s) of the OCT image formed in step S35 to the ophthalmic apparatus or the server apparatus provided outside the ophthalmic apparatus 1, and to acquire the one or more tomographic information images at the sent slice position(s).

### (S57: Perform Registration Processing)

Next, the main controller 211 controls the data processor 230 to perform the registration processing on the OCT image formed in step S55 and each of the one or more tomographic information images acquired in step S56.

For example, the data processor 230 performs the position matching between the OCT image and the tomographic information image to be processed by repeating an affine transformation on one of both of the images so as to match one or more characteristics regions or the layer regions (or the boundaries of the layer regions) commonly depicted in both of the images. Alternatively, the data processor 230 performs the position matching between the OCT image and the tomographic information image to be processed by repeating an affine transformation on one of both of the images so as to maximize the correlation value between both of the images.

### (S58: Perform Segmentation Processing)

Next, the main controller 211 controls the segmentation processor 232 to perform the segmentation processing on the OCT image formed in step S55 to identify the boundary of a desired layer region, in the same manner as in step S7.

It should be noted that the order of performing steps S57 and S58 may be interchanged.

### (S59: Display OCT Image and Tomographic Information Image)

Subsequently, the main controller 211 controls the display controller 211A to display the OCT image, in which the boundary of the desired layer region identified in step S58 is distinguishably depicted, and at least one of the one or more tomographic information images acquired in step S56 on the display unit 240A, in the same manner as in step S9.

### (S60: Perform Modification Processing)

Subsequently, the main controller 211 controls the modification processor 233 to perform the modification processing for modifying the boundary of the layer region identified in step S58 in the OCT image, in the same manner as in step S10.

### (S61: Store)

Subsequently, the main controller 211 stores the OCT image, in which the boundary of the layer region has been modified in the modification processing in step S60, in the storage unit 212, in the same manner as in step S11.

### (S62: Next Layer Region?)

Subsequently, the main controller 211 determines whether or not there is a boundary of a layer region to be modified next, in the same manner as in step S12.

When it is determined in step S62 that there is a boundary of a layer region to be modified next (S62: Y), the operation of the ophthalmic apparatus 1 proceeds to step S59. When it is determined in step S62 that there is not a boundary of a layer region to be modified next (S62: N), the operation of the ophthalmic apparatus 1 proceeds to step S63.

### (S63: Next Image?)

When it is determined in step S62 that there is not a boundary of a layer region to be modified next (S62: N), the main controller 211 determines whether or not there is an image in which a boundary of a layer region should be modified next, in the same manner as in step S12.

When it is determined in step S63 that there is an image in which a boundary of a layer region should be modified next (S63: Y), the operation of the ophthalmic apparatus 1 proceeds to step S55. After proceeding to step S55, steps S55 through S63 are performed sequentially for the OCT images at the next slice position. When it is determined in step S63 that there is not an image in which a boundary of a next region should be modified next (S63: N), the operation of the ophthalmic apparatus 1 is terminated (END).

### (Fourth Operation Example)

In the fourth operation example, the boundary of the layer region identified by performing the segmentation processing on the OCT image is manually modified while referring to the one or more tomographic information images generated inside the ophthalmic apparatus 1 and the one or more tomographic information images acquired from outside the ophthalmic apparatus 1. In this case, it is assumed that the ophthalmic apparatus 1 is capable of communicating with an external ophthalmic apparatus capable of acquiring the one or more tomographic information images via a network, or a server apparatus that stores electronic medical record information in which the one or more tomographic information images are associated with the eye to be examined.

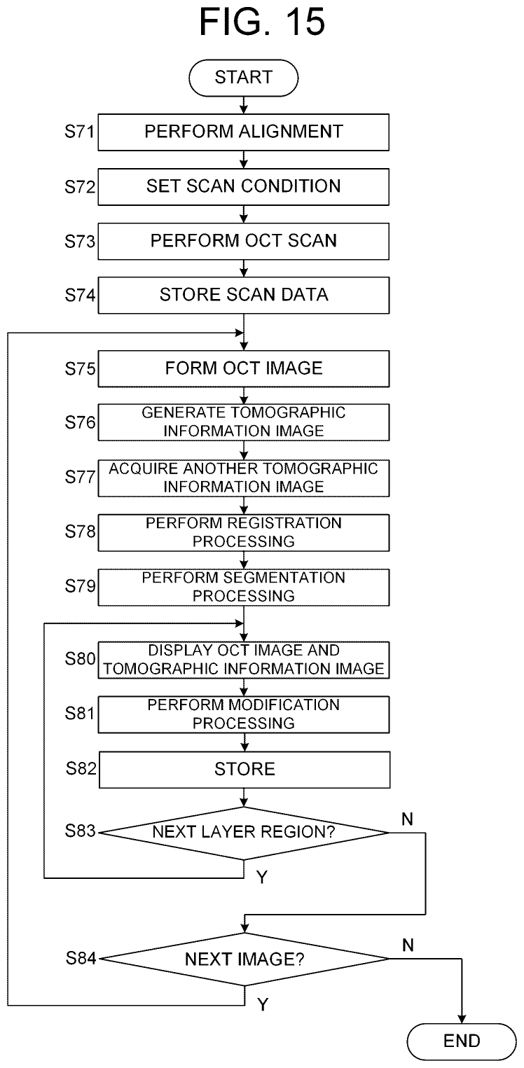

FIG. 15 shows a flow diagram of the fourth operation example of the ophthalmic apparatus 1 according to the embodiments. The storage unit 212 stores a computer program for realizing the processing shown in FIG. 15. The main controller 211 operates according to the computer program(s), and thereby the main controller 211 performs the processing shown in FIG. 15.

### (S71: Perform Alignment)

First, the main controller 211 performs alignment adjustment of the optical system relative to the eye E to be examined, in the same manner as in step S1.

### (S72: Set Scan Condition)

Next, the main controller 211 sets the scan condition(s) so as to scan a desired scan region with a desired scan mode, in the same manner as in step S2.

### (S73: Perform OCT Scan)

Subsequently, the main controller 211 controls the optical scanner 42, the OCT unit 100, and the like to perform OCT scan under the scan condition set in step S72, in the same manner as in step S3.

### (S74: Store Scan Data)

The main controller 211 stores the scan data obtained in step S73 in the storage unit 212, in the same manner as in step S4.

### (S75: Form OCT Image)

Subsequently, the main controller 211 controls the image forming unit 220 to form a single OCT image (B-scan image), which is a tomographic image at a predetermined slice position, from the scan data stored in step S74, in the same manner as in step S5.

### (S76: Generate Tomographic Information Image)

Subsequently, the main controller 211 controls the tomographic information image generator 231 to generate the one or more tomographic information images at the same slice position as the OCT image formed in step S75, based on the scan data stored in step S74 or the OCT image formed in step S75, in the same manner as in step S6.

### (S77: Acquire Tomographic Information Image)

Subsequently, the main controller 211 controls a communication unit (not shown) to acquire other one or more tomographic information images at (almost) the same slice position as the OCT image formed in step S75 from the ophthalmic apparatus or the server apparatus, the ophthalmic apparatus or the server apparatus being provided outside the ophthalmic apparatus 1, in the same manner as in step S56.

### (S78: Perform Registration Processing)

Next, the main controller 211 controls the data processor 230 to perform the registration processing on the OCT image formed in step S75 and each of the one or more tomographic information images acquired in step S77, in the same manner as in step S57.

### (S79: Perform Segmentation Processing)

Next, the main controller 211 controls the segmentation processor 232 to perform the segmentation processing on the OCT image formed in step S75 to identify the boundary of a desired layer region, in the same manner as in step S7.

### (S80: Display OCT Image and Tomographic Information Image)

Subsequently, the main controller 211 controls the display controller 211A to display the OCT image, in which the boundary of the desired layer region identified in step S78 is distinguishably depicted, the one or more tomographic information images generated in step S76, and at least one of the one or more tomographic information images acquired in step S77 on the display unit 240A, in the same manner as in step S9.

### (S81: Perform Modification Processing)

Subsequently, the main controller 211 controls the modification processor 233 to perform the modification processing for modifying the boundary of the layer region identified in step S79 in the OCT image, in the same manner as in step S10.

### (S82: Store)

Subsequently, the main controller 211 stores the OCT image, in which the boundary of the layer region has been modified in the modification processing in step S81, in the storage unit 212, in the same manner as in step S11.

### (S83: Next Layer Region?)

Subsequently, the main controller 211 determines whether or not there is a boundary of a layer region to be modified next, in the same manner as in step S12.

When it is determined in step S83 that there is a boundary of a layer region to be modified next (S83: Y), the operation of the ophthalmic apparatus 1 proceeds to step S80. When it is determined in step S83 that there is not a boundary of a layer region to be modified next (S83: N), the operation of the ophthalmic apparatus 1 proceeds to step S84.

### (S84: Next Image?)

When it is determined in step S84 that there is not a boundary of a layer region to be modified next (S84: N), the main controller 211 determines whether or not there is an image in which a boundary of a layer region should be modified next, in the same manner as in step S12.

When it is determined in step S84 that there is an image in which a boundary of a layer region should be modified next (S84: Y), the operation of the ophthalmic apparatus 1 proceeds to step S75. After proceeding to step S75, steps S75 through S84 are performed sequentially for the OCT images at the next slice position. When it is determined in step S84 that there is not an image in which a boundary of a next region should be modified next (S84: N), the operation of the ophthalmic apparatus 1 is terminated (END).

### [Actions]

The ophthalmic information processing apparatus, the ophthalmic apparatus, the ophthalmic information processing method, and the program according to the embodiments will be described.

The first aspect of the embodiments is an ophthalmic information processing apparatus (data processor 230 (and image forming unit 220)) including an acquisition unit (optical system included in the OCT unit 100, image forming unit 220, and tomographic information image generator 231, or communication unit (not shown)), a segmentation processor (232), and a display controller (211A). The acquisition unit is configured to acquire an OCT image and one or more tomographic information images. The OCT image is a tomographic image of an eye (E) to be examined. The tomographic information images are generated using a method different from a method of generating the OCT image and representing tomographic information of the eye to be examined. The segmentation processor is configured to perform segmentation processing on the OCT image to identify a boundary of a layer region in a depth direction. The display controller is configured to display the OCT image, in which the boundary identified by the segmentation processor is distinguishably depicted, and the one or more tomographic information images on a display means (display apparatus 3, display unit 240A).

According to such an aspect, the one or more tomographic information images generated using a method different from a method for generating the OCT image are displayed together with the OCT image on the display means. In the tomographic information image, the layer structures different from the layer structures depicted in the OCT image are depicted with emphasis. This allows the user to judge of necessity for modifying the boundary with high accuracy, by observing the boundary of the layer region identified in the OCT image in detail while referring to the tomographic information image.

In the second aspect of the embodiments, in the first aspect, the display controller is configured to display the OCT image, in which the boundary is depicted, and one of the one or more tomographic information images in a superimposed state on the display means.

According to such an aspect, the boundary of the layer region identified in the OCT image can be identified with high accuracy, by superimposing the tomographic information image, in which the layer structures different from the layer structures depicted in OCT image are depicted with emphasis. Thereby, the layer region in the tomographic structure of the eye to be examined can be observed in more detail, while greatly reducing labor.

In the third aspect of the embodiments, in the first aspect or the second aspect, the boundary of the layer region in the depth direction is distinguishably depicted in the one or more tomographic information images.

According to such an aspect, the boundary of the layer region depicted in the tomographic information image can be easily identified. Therefore, the layer region in the tomographic structure of the eye to be examined can be observed in more detail while greatly reducing labor.

In the fourth aspect of the embodiments, in any one of the first aspect to the third aspect, the one or more tomographic information images are a plurality of tomographic information images. The display controller is configured to display the plurality of tomographic information images in a parallel or a superimposed state on the display means.

According to such an aspect, the plurality of tomographic information images is displayed in parallel or superimposed manner on the display means together with the OCT image. Thereby, the layer region in the tomographic structure of the eye to be examined can be observed in more detail.

The fifth aspect of the embodiments, in any one of the first aspect to the fourth aspect, further includes an operation unit (240B), and a modification processor (232) configured to perform modification processing for modifying the boundary in the OCT image, based on operation information of a user to the operation unit.

According to such an aspect, the layer region in the tomographic structure of the eye to be examined can be modified with high accuracy while reducing labor.

In the sixth aspect of the embodiment, in any one of the first aspect to the fifth aspect, the one or more tomographic information images include an image generated based on the OCT image.

According to such an aspect, the boundary of the layer region can be observed in detail while associating the OCT image and the tomographic information images, without performing registration processing.

In the seventh aspect of the embodiments, in any one of the first aspect to the sixth aspect, the one or more tomographic information images include at least one of an OCT angiography image, an attenuation coefficient image, a polarization information image (DOPU image), or (and) a birefringence image.

According to such an aspect, the necessity for modifying the boundary can be judged with high accuracy, by observing the boundary of the layer region identified in the OCT image in detail while referring to at least one of the OCTA image, the attenuation coefficient image, the polarization information image, or (and) the birefringence image.

In the eighth aspect of the embodiments, in any one of the first aspect to the seventh aspect, the one or more tomographic information images include a first tomographic information image and a second tomographic information image different from the first tomographic information image. The display controller is configured to display the OCT image, in which a boundary of a first layer region is depicted, and the first tomographic information image on the display means, and to display the OCT image, in which a boundary of a second layer region different from the first layer region is depicted, and the second tomographic information image on the display means.

According to such an aspect, the necessity for modifying the boundary can be judged with high accuracy, by observing the boundaries of the layer regions in the OCT image in detail while referring to the tomographic information images.

The ninth aspect of the embodiments is an ophthalmic apparatus (1) including an optical system (optical system included in the OCT unit 100) configured to perform OCT on an eye to be examined, an image forming unit (220) configured to form an OCT image based on a detection result of interference light acquired by the optical system; and, the ophthalmic information processing apparatus of any one of the first aspect to the eighth aspect.

According to such an aspect, the one or more tomographic information images generated using a method different from a method for generating the OCT image are displayed together with the OCT image on the display means. In the tomographic information image, the layer structures different from the layer structures depicted in the OCT image are depicted with emphasis. This allows to provide the ophthalmic apparatus capable of judging the necessity for modifying the boundary with high accuracy, by observing the boundary of the layer region identified in the OCT image in detail while referring to the tomographic information image.

The tenth aspect of the embodiments, in the ninth aspect, further includes a tomographic information image generator (231) configured to generate at least one of the one or more tomographic information images based on the OCT image.

According to such an aspect, the necessity for modifying the boundary can be judged with high accuracy, by observing the boundary of the layer region identified in the OCT image in detail while referring to the tomographic information image, without performing registration processing between the OCT image and the tomographic information image.

The eleventh aspect of the embodiments is an ophthalmic information processing method includes an acquisition step, a segmentation processing step, and a display control step. The acquisition step is performed to acquire an OCT image and one or more tomographic information images. The OCT image is a tomographic image of an eye (E) to be examined. The tomographic information images are generated using a method different from a method of generating the OCT image and representing tomographic information of the eye to be examined. The segmentation processing step is performed to perform segmentation processing on the OCT image to identify a boundary of a layer region in a depth direction. The display control step is performed to display the OCT image, in which the boundary identified by the segmentation processor is distinguishably depicted, and the one or more tomographic information images on a display means (display apparatus 3, display unit 240A).

According to such an aspect, the one or more tomographic information images generated using a method different from a method for generating the OCT image are displayed together with the OCT image on the display means. In the tomographic information image, the layer structures different from the layer structures depicted in the OCT image are depicted with emphasis. This allows the user to judge of necessity for modifying the boundary with high accuracy, by observing the boundary of the layer region identified in the OCT image in detail while referring to the tomographic information image.

In the twelfth aspect of the embodiments, in the eleventh aspect, the display control step is performed to display the OCT image, in which the boundary is depicted, and one of the one or more tomographic information images in a superimposed state on the display means.

According to such an aspect, the boundary of the layer region identified in the OCT image can be modified, by superimposing the tomographic information image, in which the layer structures different from the layer structures depicted in OCT image are depicted with emphasis. Thereby, the layer region in the tomographic structure of the eye to be examined can be observed in more detail, while greatly reducing labor.

In the thirteenth aspect of the embodiments, in the eleventh aspect or the twelfth aspect, the boundary of the layer region in the depth direction is distinguishably depicted in the one or more tomographic information images.

According to such an aspect, the boundary of the layer region depicted in the tomographic information image can be easily identified. Therefore, the layer region in the tomographic structure of the eye to be examined can be observed in more detail while greatly reducing labor.

In the fourteenth aspect of the embodiments, in any one of the eleventh aspect to the thirteenth aspect, the one or more tomographic information images are a plurality of tomographic information images. The display control step is performed to display the plurality of tomographic information images in a parallel or a superimposed state on the display means.

According to such an aspect, the plurality of tomographic information images is displayed in parallel or superimposed manner on the display means together with the OCT image. Thereby, the layer region in the tomographic structure of the eye to be examined can be observed in more detail.

The fifteenth aspect of the embodiments, in any one of the eleventh aspect to the fourteenth aspect, further includes a modification processing step of performing modification processing for modifying the boundary in the OCT image, based on operation information of a user to an operation unit (240B).

According to such an aspect, the layer region in the tomographic structure of the eye to be examined can be modified with high accuracy while reducing labor.

In the sixteenth aspect of the embodiments, in any one of the eleventh aspect to the fifteenth aspect, the one or more tomographic information images include an image generated based on the OCT image.

According to such an aspect, the boundary of the layer region can be observed in detail while associating the OCT image and the tomographic information images, without performing registration processing.

In the seventeenth aspect of the embodiments, in any one of the eleventh aspect to the sixteenth aspect, the one or more tomographic information images include at least one of an OCT angiography image, an attenuation coefficient image, a polarization information image (DOPU image), or (and) a birefringence image.

According to such an aspect, the necessity for modifying the boundary can be judged with high accuracy, by observing the boundary of the layer region identified in the OCT image in detail while referring to at least one of the OCTA image, the attenuation coefficient image, the polarization information image, or (and) the birefringence image.

In the eighteenth aspect of the embodiments, in any one of the eleventh aspect to the seventeenth aspect, the one or more tomographic information images include a first tomographic information image and a second tomographic information image different from the first tomographic information image. The display control step is performed to display the OCT image, in which a boundary of a first layer region is depicted, and the first tomographic information image on the display means, and to display the OCT image, in which a boundary of a second layer region different from the first layer region is depicted, and the second tomographic information image on the display means.

According to such an aspect, the necessity for modifying the boundary can be judged with high accuracy, by observing the boundaries of the layer regions in the OCT image in detail while referring to the tomographic information images.

The nineteenth aspect of the embodiments is a program of causing a computer to execute each step of the ophthalmic information processing method of any one of the eleventh aspect to the eighteenth aspect.

According to such an aspect, this allows to provide the program capable of judging the necessity for modifying the boundary with high accuracy, by observing the boundary of the layer region identified in the OCT image in detail while referring to the tomographic information image.

The configuration described above is only an example for suitably implementing the present invention. Therefore, any modification (omission, substitution, addition, etc.) within the scope of the gist of the present invention can be appropriately applied. The configuration to be employed is selected according to the purpose, for example. In addition, depending on the configuration to be applied, it is possible to obtain the actions and effects obvious to those skilled in the art and the actions and effects described in this specification.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ophthalmic information processing apparatus, comprising:
processing circuitry configured as
acquisition circuitry configured to acquire an OCT image and one or more tomographic information images, the OCT image being a tomographic image of an eye to be examined, the tomographic information images being generated using a method different from a method of generating the OCT image and representing tomographic information of the eye to be examined;
a segmentation processor configured to perform segmentation processing on the OCT image to identify a boundary of a layer region in a depth direction; and
a display controller configured to display the OCT image, in which the boundary identified by the segmentation processor is distinguishably depicted, and the one or more tomographic information images on a display.

2. The ophthalmic information processing apparatus of claim 1, wherein
the display controller is configured to display the OCT image, in which the boundary is depicted, and one of the one or more tomographic information images in a superimposed state on the display.

3. The ophthalmic information processing apparatus of claim 1, wherein
the boundary of the layer region in the depth direction is distinguishably depicted in the one or more tomographic information images.

4. The ophthalmic information processing apparatus of claim 1, wherein
the one or more tomographic information images are a plurality of tomographic information images, and
the display controller is configured to display the plurality of tomographic information images in a parallel or a superimposed state on the display.

5. The ophthalmic information processing apparatus of claim 1, wherein the processing circuitry is further configured as:
operation circuitry; and
a modification processor configured to perform modification processing for modifying the boundary in the OCT image, based on operation information of a user provided to the operation circuitry.

6. The ophthalmic information processing apparatus of claim 5, wherein
the one or more tomographic information images include an image generated based on the OCT image.

7. The ophthalmic information processing apparatus of claim 6, wherein
the one or more tomographic information images include at least one of an OCT angiography image, an attenuation coefficient image, a polarization information image, or a birefringence image.

8. The ophthalmic information processing apparatus of claim 5, wherein
the one or more tomographic information images include a first tomographic information image and a second tomographic information image different from the first tomographic information image, and
the display controller is configured to display the OCT image, in which a boundary of a first layer region is depicted, and the first tomographic information image on the display means, and to display the OCT image, in which a boundary of a second layer region different from the first layer region is depicted, and the second tomographic information image on the display.

9. An ophthalmic apparatus, comprising:
an optical system configured to perform OCT on an eye to be examined;
an image forming circuit configured to form an OCT image based on a detection result of interference light acquired by the optical system; and
an ophthalmic information processing apparatus, wherein
the ophthalmic information processing apparatus includes processing circuitry configured as:
acquisition circuitry configured to acquire the OCT image and one or more tomographic information images, the OCT image being a tomographic image of an eye to be examined, the tomographic information images being generated using a method different from a method of generating the OCT image and representing tomographic information of the eye to be examined;
a segmentation processor configured to perform segmentation processing on the OCT image to identify a boundary of a layer region in a depth direction;
a display controller configured to display the OCT image, in which the boundary identified by the segmentation processor is distinguishably depicted, and the one or more tomographic information images on a display;
operation circuitry; and
a modification processor configured to perform modification processing for modifying the boundary in the OCT image, based on operation information of a user provided to the operation circuitry.

10. The ophthalmic apparatus of claim 9, further comprising
a tomographic information image generator configured to generate at least one of the one or more tomographic information images based on the OCT image.

11. An ophthalmic information processing method, comprising:
acquiring an OCT image and one or more tomographic information images, the OCT image being a tomographic image of an eye to be examined, the tomographic information images being generated using a method different from a method of generating the OCT image and representing tomographic information of the eye to be examined;
performing segmentation processing on the OCT image to identify a boundary of a layer region in a depth direction; and
controlling display of the OCT image, in which the boundary identified by the segmentation processor is distinguishably depicted, and the one or more tomographic information images on a display.

12. The ophthalmic information processing method of claim 11, wherein
the controlling display is performed to display the OCT image, in which the boundary is depicted, and one of the one or more tomographic information images in a superimposed state on the display.

13. The ophthalmic information processing method of claim 11, wherein
the boundary of the layer region in the depth direction is distinguishably depicted in the one or more tomographic information images.

14. The ophthalmic information processing method of claim 11, wherein
the one or more tomographic information images are a plurality of tomographic information images, and
the controlling display is performed to display the plurality of tomographic information images in a parallel or a superimposed state on the display.

15. The ophthalmic information processing method of claim 11, further comprising
performing modification processing for modifying the boundary in the OCT image, based on operation information of a user provided to operation circuitry.

16. The ophthalmic information processing method of claim 15, wherein
the one or more tomographic information images include an image generated based on the OCT image.

17. The ophthalmic information processing method of claim 16, wherein
the one or more tomographic information images include at least one of an OCT angiography image, an attenuation coefficient image, a polarization information image, or a birefringence image.

18. The ophthalmic information processing method of claim 15, wherein
the one or more tomographic information images include a first tomographic information image and a second tomographic information image different from the first tomographic information image, and
the controlling display is performed to display the OCT image, in which a boundary of a first layer region is depicted, and the first tomographic information image on the display, and to display the OCT image, in which a boundary of a second layer region different from the first layer region is depicted, and the second tomographic information image on the display.

19. A computer readable non-transitory recording medium in which a program for causing a computer to execute each step of an ophthalmic information processing method is recorded, wherein
the ophthalmic information processing method comprising:
acquiring an OCT image and one or more tomographic information images, the OCT image being a tomographic image of an eye to be examined, the tomographic information images being generated using a method different from a method of generating the OCT image and representing tomographic information of the eye to be examined;
performing segmentation processing on the OCT image to identify a boundary of a layer region in a depth direction;
controlling display of the OCT image, in which the boundary identified by the segmentation processor is distinguishably depicted, and the one or more tomographic information images on a display; and
performing modification processing for modifying the boundary in the OCT image, based on operation information of a user provided to operation circuitry.
